SEQUENCE LISTING (8 sequences)

SEQ ID NO: 1 - protein, 7 aa, Artificial Sequence
  Description of Artificial Sequence Synthetic peptide
  HK-7 (1-7)
HLLQFNK

SEQ ID NO: 2 - protein, 18 aa, Artificial Sequence
  Description of Artificial Sequence Synthetic peptide
  NQ-18 (16-33)
NAVPFYAFYGCYCGWGGQ

SEQ ID NO: 3 - protein, 9 aa, Artificial Sequence
  Description of Artificial Sequence Synthetic peptide
  NS-9 (105-113)
NGYMFYPDS

SEQ ID NO: 4 - protein, 13 aa, Artificial Sequence
  Description of Artificial Sequence Synthetic peptide
  NG-13 (105-117)
NGYMFYPDSRCRG

SEQ ID NO: 5 - protein, 20 aa, Artificial Sequence
  Description of Artificial Sequence Synthetic peptide
  NR-20 (16-35)
NAVPFYAFYGCYSGWGGQGR

SEQ ID NO: 6 - protein, 13 aa, Artificial Sequence
  Description of Artificial Sequence Synthetic peptide
  HK-13 (1-13)
HLLQFNKMIKFET

SEQ ID NO: 7 - protein, 122 aa, Artificial Sequence
  Description of Artificial Sequence Synthetic polypeptide
  CBb unit
HLLQFNKMIKFETRKNAVPFYAFYGCYCGWGGQGRPKDATDRCCFVHDCCYGKLAKCNTKWDIYRYSLKSGYITCGKGTWCEEQICECDRVAAECLRRSLSTYKNGYMFYPDSRCRGPSETC

SEQ ID NO: 8 - protein, 6 aa, Artificial Sequence
  Description of Artificial Sequence Synthetic 6xHis tag
HHHHHH

(19) United States
(12) Patent Application Publication
(10) Pub. No.: US 2019/0391160 A1
(43) Pub. Date: Dec. 26, 2019

(54) METHODS AND COMPOSITIONS FOR MODIFYING CYSTIC FIBROSIS TRANSMEMBRANE CONDUCTANCE REGULATOR ACTIVITY

(71) Applicants: INSTITUT PASTEUR, Paris (FR); INSTITUT NATIONAL DE LA SANTE ET DE LA RECHERCHE MEDICALE, Paris (FR); INSTYTUT BIOCHEMII I BIOFIZYKI POLSKIEJ AKADEMII NAUK, Warszawa (PL)

(72) Inventors: Norbert ODOLCZYK, WARSZAWA (PL); Aleksander EDELMAN, CHATENAY-MALABRY (FR); Piotr ZIELENKIEWICZ, WARSZAWA (PL); Grazyna FAURE-KUZMINSKA, NOISY LE GRAND (FR)

(21) Appl. No.: 16/097,293

(22) PCT Filed: Apr. 28, 2017
(86) PCT No.: PCT/IB2017/000690

§ 371 Date: Oct. 29, 2018

Related U.S. Application Data
(60) Provisional application No. 62/329,875, filed on Apr. 29, 2016.

(51) Int. Cl. G01N 33/68; A61P 11/00; C07K 14/46

(52) U.S. Cl. CPC G01N 33/6872; A61P 11/00; A61K 38/00; G01N 2500/10; C07K 14/46

(57) ABSTRACT

Methods of increasing CFTR activity in a cell, comprising contacting the cell with a peptide modulator of CFTR to thereby increase CFTR activity in the cell. Methods of treating cystic fibrosis in a subject in need thereof, comprising administering an effective amount of a peptide modulator of CFTR to the subject to thereby increase CFTR activity in the subject. Pharmaceutical compositions comprising a peptide modulator of CFTR. The peptide modulator may comprise or consist of an amino acid fragment of the CB subunit of crotoxin from Crotalus durrissus terrificus venom. The peptide modulator may comprise or consist of the amino acid sequence of SEQ ID NOS: 1, 2, 3, 4, 5 or 6.

### SEQUENCE LISTING

The instant application contains a Sequence Listing which has been filed electronically in ASCII format and is hereby incorporated by reference in its entirety. Said ASCII copy, created on Jul. 31, 2019, is named B12425_SL.txt and is 3,672 bytes in size.

### INTRODUCTION

Cystic fibrosis (CF) is caused by mutations in the CFTR gene, which encodes a 1480-amino acid transmembrane protein with a symmetrical structure composed of two membrane-spanning domains (MSD1 and MSD2), each with six transmembrane helices, and two nucleotide binding domains (NBD1 and NBD2) separated by a hydrophilic regulatory domain (R)¹. The Cystic Fibrosis Transmembrane conductance Regulator (CFTR) is a unique chloride (Cl—) channel that links ATP hydrolysis to channel gating and regulates transepithelial fluid transport^{2,3}. A deletion of phenylalanine at position 508 (DF508) in the NBD1 domain is present in at least one allele in 90% of patients suffering from cystic fibrosis and gives rise to an incorrectly folded protein which is rapidly degraded and cannot reach the plasma membrane⁴. This defect leads to reduced intrinsic Cl— membrane channel conductance in CF cells, compared with wild type CFTR⁵.

A number of studies have been conducted to search for a pharmacological approach to correct the dysfunction of the mutated proteins⁶. For the DF508 mutation, small molecule compounds have been developed to facilitate trafficking and delivery of the abnormal protein to the plasma membrane (correctors) and to improve channel gating (potentiators). To this end, two strategies have been employed. The first is to stabilize ΔF508CFTR using a high-throughput screening approach to identify compounds that are able to correct ΔF508CFTR dysfunction. The best examples are the corrector VX-809 and the potentiator VX-770 (ivacaftor), the latter being the most successful example of this approach. Today Ivacaftor is used to treat G551D CF patients, but neither VX-809 nor ivacaftor are sufficiently active in ΔF508 CF patients. The second strategy is a hypothesis-driven approach, which has led to the identification of correctors such as curcumin and resveratrol derived from plants Z. This strategy also suggested that the site of interaction between cytokeratin-8 (K8) and DF508CFTR should constitute a therapeutic target 2. However, there is still a major need for the development of new selective and high affinity compounds acting as dual modulators. The inventions disclosed herein meet these and other needs.

### SUMMARY

In an attempt to identify a new class of correctors/modulators of ΔF508 CFTR natural, multifunctional proteins present in snake venom were studied. In particular, the phospholipase A₂ (PLA₂) CB subunit of crotoxin from the South American rattlesnake Crotalus durissus terrificus was investigated. During investigation of the cytokeratin 8-NBD1 interaction¹⁰ and using CB as a negative control, a surprisingly high binding affinity of CB for NBD1 was discovered. Viperidae snake venom PLA₂ (structurally homologous to inflammatory, non-pancreatic human sPLA₂-IIA) are known to possess a large spectrum of pharmacological functions. However, no effect on CFTR was taught or suggested in the art. Numerous studies have shown that neurotoxic phospholipases A₂ can enter into cells and interact with various protein targets, exhibiting different pharmacological effects, sometimes independently of their enzymatic activity^{11 12 13 14 15}. In particular, crotoxin from the South American rattlesnake Crotalus durissus terrificus, a heterodimeric CA-CB presynaptic toxin with PLA₂ activity¹⁶ exhibits bactericidal, antiviral, anti-cytotoxic properties against various tumor cells and can also regulate Ca²⁺ channel currents^{17; 18; 19; 20}. Ollivier-Bousquet et al²¹ showed that the CB subunit of crotoxin, alone or in combination with CA, was able to adsorb onto the membrane of epithelial cells and to be internalized to induce lectin secretion. More recently Lomeo et al²⁰ reported that the CB subunit of crotoxin is internalized within less than 5 min in cerebellar granule cells and that CB internalization does not depend on the presence of CA and does not depend on PLA₂ activity. Both subunits of crotoxin exist in four major natural isoforms (acidic CA₁₋₄ and basic CBa₂/b/c/d) and represent interesting models to identify new PLA₂-binding targets^{14; 16}. None of these studies pointed to a role for crotoxin in modulating the molecular mechanisms that underly cystic fibrosis.

The interaction of the CB subunit of crotoxin with CFTR and the potential effect of CB on Cl⁻ channel activity was investigated. Experimental evidence is provided that CB directly interacts with the wild-type (WT) and mutated NBD1 domain of human CFTR and corrects the functional defect of ΔF508CFTR. It is shown that CB behaves as a dual modulator of CFTR activity as a potentiator, increasing the Cl⁻ channel current, and as a corrector, facilitating transport and insertion of DF508CFTR into the plasma membrane.

Accordingly, in a first aspect this disclosure provides methods of increasing CFTR activity in a cell, comprising contacting the cell with a peptide modulator of CFTR to thereby increase CFTR activity in the cell; wherein the peptide modulator comprises or consists of an amino acid fragment of the CB subunit of crotoxin from Crotalus durrissus terrificus venom. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

In another aspect this disclosure provides methods of treating cystic fibrosis in a subject in need thereof, comprising administering an effective amount of a peptide modulator of CFTR to the subject to thereby increase CFTR activity in the subject; wherein the peptide modulator comprises or consists of an amino acid fragment of the CB subunit of crotoxin from Crotalus durrissus terrificus venom. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and a polypeptide comprising the amino acid sequence NGYMFYPDS (SEQ ID NO: 3), a polypeptide consisting of the amino acid sequence SEQ ID NO: 3, a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

In another aspect this disclosure provides pharmaceutical compositions comprising a peptide modulator of CFTR; wherein the peptide modulator comprises or consists of an amino acid fragment of the CB subunit of crotoxin from Crotalus durrissus terrificus venom. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, a polypeptide comprising a functional variant of SEQ ID NO: 2; and a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

In another aspect this disclosure provides uses of a peptide modulator of CFTR for the manufacture of a medicament for use in treating cystic fibrosis; wherein the peptide modulator comprises or consists of an amino acid fragment of the CB subunit of crotoxin from Crotalus durrissus terrificus venom.

In another aspect this disclosure provides peptide modulators of CFTR for use in treating cystic fibrosis; wherein the peptide modulator comprises or consists of an amino acid fragment of the CB subunit of crotoxin from Crotalus durrissus terrificus venom.

In another aspect this disclosure provides methods of characterizing a CFTR modulator. In some embodiments the methods comprise contacting a cell that expresses CFTR with a peptide modulator of CFTR that increases CFTR activity in the cell; contacting the cell with a candidate agent; and determining whether the candidate agent modulates the effect of the peptide modulator of CFTR on CFTR activity; wherein the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and a polypeptide comprising the amino acid sequence NGYMFYPDS (SEQ ID NO: 3), a polypeptide consisting of the amino acid sequence SEQ ID NO: 3, a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the candidate agent modulates the effect of the peptide modulator of CFTR on CFTR activity and the candidate agent is identified as a CFTR modulator.

### BRIEF DESCRIPTION OF THE DRAWINGS

FIGS. 1A to 1D show SPR-direct binding of CB to human NBD1 domain of CFTR and potentiating effect on CFTR-Cl⁻ channel current by patch clamp experiments in Xenopus laevis oocytes. A. SPR interaction of isoform CBa₂ (injected at concentrations of 20, 10, 5, 2.5, 1.25 mg/ml) with immobilized hNBD1. B. SPR binding of isoform CBc (injected at concentrations of 10, 5, 2.5, 1.25 mg/ml), with immobilized hNBD1. C. The potentiating effect of CBa₂ on CFTR. Current-voltage I/V relationships were determined in CFTR-expressing oocytes injected with 1 ng of CFTR cRNA and in oocytes expressing CFTR and supplemented with 0.5 ng CBa₂, before and after superfusion with PKA-activating cocktail (1 μM forskolin plus 100 μM IBMX). Error bars represent the standard error of the mean for each data point (n=8). Black circles correspond to I/V curves obtained for oocytes expressing CFTR alone; white circles for oocytes expressing CFTR and injected with CBa₂; green lines correspond to I/V curves obtained in the presence of PKA-activating cocktail; red lines to I/V curves obtained in the presence of 10 μM of Inh-172; black lines to the I/V curves obtained in control conditions (without PKA-activating cocktail). Results are shown as means±SEM, with n as the number of oocytes from different donors (n=8), N=3. D. Summary of the results presented in FIG. 1C at −100 and +40 mV. 10 μM Inh-172 was used. Differences between the same experimental conditions +/−PKA-activating cocktail were always p<0.05. E. Immunoblot of CFTR in microsomal proteins from CFTR-expressing oocytes (control) and CFTR with CBa₂. Oocytes were injected with 1 ng CFTR cRNA or 50 nl water (control oocytes). Microsomal proteins were separated by SDS-PAGE, blotted to nitrocellulose and revealed by anti MM13.4 anti-CFTR⁴⁶. In each panel, lanes are as follows: 1: water-injected alone; 2: water-injected+CBa₂; 3: CFTR alone and 4: CFTR+CBa₂-injected.

FIGS. 2A to 2D show the potentiating effect of CB shown by different patch-clamp experiments in HeLa cells and ex vivo in mouse colon tissues. A. Current recordings of CFTR channel activity on the same cell-excised, inside-out membrane patch clamped at −60 mV. The patch was bathed in symmetrical high-Cl solution in the presence of ATP-Mg+PKA, ATP-Mg+PKA+CB and ATP-Mg+PKA+CB+Inh172+Glibenclamide in the bath. The ATP concentration at the intracellular side of the membrane patch was 1 mM. C, current level corresponding to closure of all CFTR channels. Insets: 1, 2 and 3, excerpts at an expanded time scale (*) taken from the indicated sections of the traces. B. Effects of CB on CFTR channel activity. The ATP concentration at the intracellular side of the membrane patch was 1 mM. Values are means±SEM. *, p<0.05 versus ATP-Mg+PKA. Current recordings show that addition of 1 nM CB increases channel activity by a factor of 2.3. C. Ex vivo measurements in mouse colon tissue show that CB increases c-AMP-dependent Cl⁻ short-circuit current (DIsc) changes. ΔIsc (Y axis) was recorded as a function of time (X axis). After stabilization of Isc, the amiloride (100 μM) was added to the apical side to block the Na⁺ current, the cAMP-dependent Cl⁻ conductance was activated by addition of forskolin (1 μM) plus IBMX (100 μM). After stabilization of ΔIsc, two concentrations of CB (0.1 μM and 1 μM) were tested. The specificity of the current was tested by addition of bumetanide (100 mM), NaK₂Cl inhibitor. D. Potentiating effect of CB on ΔF508-CFTR pretreated with Corr-4A corrector. ΔF508-CFTR expressing HeLa Cells were treated with 1 μM Corr4a for 24 h to facilitate transport and insertion of ΔF508CFTR to the plasma membrane. Then the nystatin-perforated whole cell currents were recorded. cAMP-dependent currents were induced by addition of cAMP cocktail, I_{cAMP}. The effect of CB (InM) was then tested. The graph represents the amplitude of currents for individual cells, represented as box chart with SEM, measured at −60 mV in the following conditions: basal current, I_{cAMP} (cAMP), I_{cAMP} plus CB (1 nM CB), and the effect of inhibitors. A significant difference (p<0.05) was calculated for n=6 cells between I_{cAMP}+/−SEM (−26.8+/−5.3 pA/pF) and I_{cAMP}+CB+/−SEM (−57.2+/−11.3 pA/pF).

FIGS. 3A to 3E show direct binding of CB to human ΔF508-NBD1 and correcting effect on ΔF508-CFTR channel current by different patch clamp experiments. A. SPR interaction of isoform CBa₂ (injected at concentrations of 10, 5, 2.5, 1.25 μg/ml) with immobilized ΔF508-hNBD1 showing that CB binds to ΔF508-hNBD1. B. The potentiating effect of CBa₂ on ΔF508-CFTR. I/V relationships were determined in Δ508-CFTR expressing oocytes and in oocytes expressing ΔF508-CFTR and injected with 1 ng CBa₂ before and after exposure with PKA-activating cocktail, (symbols and lines are as in Fig. S1B). Results are means±SEM, (n=8). C. Increased expression of the mature form of CFTR in oocytes co-injected with ΔF508-CFTR and CB. D. Rescue of CFTR activity in CB treated HeLa cells for 24 hours. The cells were pretreated for 24 h with CB (1 nM) and I_{CFTR} was evaluated by whole-cell measurements nystatin-perforated patch-clamp. The amplitude of the currents is presented as basal currents (white bars), I_{CFTR} (black bars) and the amplitude of the current after Inh172 inhibition (dashed bars). The fraction of I_{CFTR}±SEM is indicated. ΔI/C=−4.6±0.19 pA/pF, n=3 non treated (−CB) and ΔI/C=−23.6±3.25 pA/pF, n=4 treated with CB (+CB), p=0.008. E. Immunoblot analysis of ΔF508-CFTR and WTCFTR in HeLa cells treated as in A. Increase in fully glycosylated CFTR (band C) was detected (left panel), no change of expression of WTCFTR was detected (right panel), n=3 independent experiments.

FIGS. 4A to 4C show the 3D molecular model of the complex between CBb and ΔF508-NBD1 of CFTR. A. The model of the complex CBb and ΔF508-NBD1 is shown as a solvent accessible surface area (SASA) calculated according to the Connolly algorithm. The surfaces of CBb and ΔF508-NBD1 are colored pink and light blue respectively, whereas the interface for both domains is highlighted as magenta for CBb and dark blue for ΔF508-NBD1. B and C. Figures B and C provide a ribbon representation of the ΔF508-NBD1 domain and CBb, respectively. Residues participating in the binding interface are indicated.

FIGS. 5A to 5H show HDX-MS analysis of the changes in stability in WTNBD1 and ΔFNBD1, accompanying the binding of CB. The % deuterium uptake in the peptides of WTNBD1 (A, C) and ΔFNBD1 (B, D) in their unbound (A, B) and bound states (C, D) after 10 s (black) and 1 min (orange) of exchange. The error bars in A-D represent the ranges of duplicate measurements. Panels E,F show differences in the fraction of exchange (% difference in deuteration between unbound and bound form) measured before and after addition of CB subunit to WTNBD1 (E) and ΔF508NBD1 (F). Fragments that become more protected upon the formation of the complex are boxed with the same color in panels E,F as their corresponding regions overlaid on NBD1 structure (PDB ID: 2BBO) for WTNBD1 (G) and ΔF508NBD1 (H), namely: red—ABCβ subdomain, blue—Structurally Diverse Region (SDR), magenta—Walker B loop, yellow—region covered within the F1-like ATP binding core subdomain and the RE domain. Additionally, the F508 residue is shown as cyan sticks representation for WTNBD1. In panels G, H the residues of the binding interface participating in interactions with the CBb subunit are shown as spheres.

FIGS. 6A-6B show possible mechanism of the interaction of CB-CFTR:CB interrupts the K8-ΔF508-CFTR pathogenic complex. A. SPR experiments show that CB prevents formation of a protein complex between K8 and ΔF508-NBD1. In control experiments, K8 binds to ΔF508-NBD1 with nanomolar affinity [10]. When CB (20 μg/ml) is bound first, K8 (200 μg/ml) cannot interact with NBD1 showing that both proteins interact with NBD1 at similar region(s). B. Schematic model explaining how the CB-ΔF508CFTR complex modifies trafficking and activity of the abnormally folded CFTR channel. ΔF508CFTR protein in the endoplasmic reticulum (ER) interacts with keratin 8 and is primed for the degradation pathway that ends in the proteasome ¹⁵[15]⁴. SPR competition experiments (FIG. 6A) showed that CB binds to ΔF508-NBD1 preventing the interaction with K8 which allows ΔF508CFTR to escape from degradation and be delivered to the plasma membrane (correcting effect, middle panel). In case of WTCFTR, CB restores Cl⁻ permeability by increasing the CFTR Cl⁻ channel current in the plasma membrane (potentiating effect, right panel). NBD1 is shown in blue, ΔF508NBD1 in red, CB in green and K8 in yellow.

FIGS. 7A to 7C show the effect of crotoxin (CA-CB complex) on CFTR. (A) Surface Plasmon Resonance (SPR) measurements show the direct binding of crotoxin to hNBD1 covalently immobilized on the Biacore sensor chip. The measurements were corrected for non-specific binding by subtraction of curves obtained by injection of the same protein solution through a blank channel (without hNBD1) on the same sensor chip. The black curve shows the result for the running buffer alone. Analyte was injected for 60 s for the association phase. This was followed by injection of the running buffer alone at the same flow-rate to trigger the dissociation phase. The response in resonance units (RU) is plotted as a function of time (in s). CA-CB at concentrations 20 mg/ml (blue); 10 mg/ml (red); 5 mg/ml (indigo); 2.5 mg/ml (green); 1.25 mg/ml (brown).

(B) Effect of CA-CB on CFTR-Cl⁻ current in X. laevis oocytes. Current-voltage (I/V) relationships were determined in CFTR-expressing oocytes injected with 1 ng of CFTR cRNA and in CFTR-expressing oocytes injected with 2.5 ng CA-CB, before and after superfusion with PKA-activating cocktail (1 μM forskolin plus 100 μM IBMX). Error bars represent the standard error of the mean for each data point (n=8). Black circles correspond to I/V curves obtained for oocytes expressing CFTR alone; white circles for oocytes expressing CFTR and injected with CACB; green lines correspond to I/V curves obtained in the presence of PKA-activating cocktail; red lines to I/V curves obtained in the presence of 10 μM of Inh-172; black lines to the I/V curves obtained in control conditions (without PKA-activating cocktail). Results are shown as means±SEM, with n as the number of oocytes from different donors (n=8), N=3.

(C) Summary of the results obtained in Figure S1B. at −100 mV and +40 mV in the CFTR—expressing oocytes. ND96, Ringer's solution. The experimental conditions are indicated above or below the bars. 10 μM Inh-172 was used.

FIGS. 8A to 8C show the effect of acidic CA subunit of crotoxin on CFTR. (A) SPR interaction of CA with immobilized hNBD1. No binding of the CA subunit (injected at the concentration 50 mg/ml) was observed. (B) Effect of CA on CFTR-Cl⁻ current in X oocytes. Current-voltage (I/V) relationships were determined in oocytes expressing CFTR alone (as indicated in Figure S1B), and in oocytes expressing CFTR and co-injected with 0.5 ng CA, before and after superfusion of oocytes with PKA-activating cocktail (1 μM forskolin plus 100 μM IBMX). White circles correspond to experiments with oocytes expressing CFTR alone; green line—to experiments in the presence of PKA-activating cocktail; white line—to experiments in basal conditions. No significant differences were observed. Results were shown as means±SEM, (n=8). (C) SPR interaction of sPLA₂-IB with hNBD1. No binding of sPLA₂-IB (injected at 20 □g/ml) was observed.

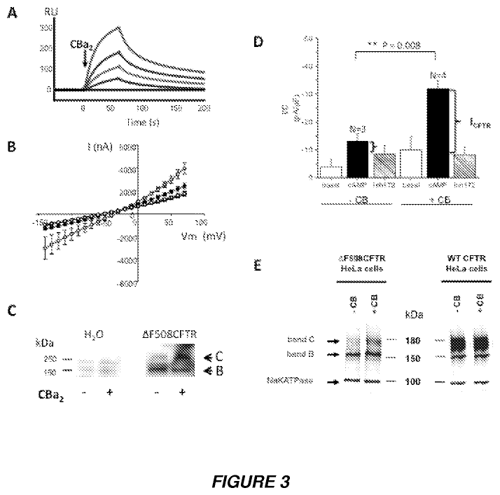

FIGS. 9A to 9C show human sPLA₂-IIA directly binds to hNBD1 and increases CFTR-Cl⁻ channel current. (A) SPR interaction of hsPLA₂-IIA (injected at concentrations of 4, 2, 1, 0.5, 0.25 mg/ml) with immobilized hNBD1. (B) The potentiating effect of hsPLA₂-IIA on CFTR. I/V relationships were determined in oocytes expressing CFTR alone and in oocytes expressing CFTR and injected with 0.5 ng hsPLA₂-IIA, (symbols as in Figure S1B). Results as means±SEM, (n=8). (C) Summary of the results obtained in FIG. 3B at −100 and +40 mV.

FIGS. 10A to 10C show human sPLA₂-IIA directly binds to ΔF508-NBD1 and inhibits Cl⁻ channel current in ΔF508-CFTR. (A) SPR interaction of hsPLA₂-IIA (injected at concentrations of 2, 1, 0.5, 0.25 μg/ml) with immobilized ΔF508-hNBD1. (B) The inhibiting effect of hsPLA₂ on ΔF508-CFTR. I/V relationships determined in ΔF508-CFTR-expressing oocytes and in oocytes expressing ΔF508-CFTR injected with hsPLA₂-IIA, (symbols as in Figure S2B). (C) Summary of the results obtained in Figure S4B. at −100 and +40 mV.

FIGS. 11A and 11B show and analysis of whether the potentiating effect of CB on CFTR Cl⁻ channel current independent of the catalytic activity of CB. (A) Effect of arachidonic acid on the CFTR-Cl⁻ current. I/V curves obtained in CFTR-expressing oocytes in the presence (white circles) and absence (black circles) of 4 μM arachidonic acid. Results are shown as means±SEM, (n=8). Arachidonic acid had no effect on I_{cAMP}. (B) Summary of the results presented in Figure S5A at −100 and +40 mV.

FIGS. 12A to 12C show results of a search for the binding interface between CB and NBD1 by SPR competition experiments and by spectrofluorimetric assay using the PLA₂-inhibitor, PMS 1062. (A) SPR studies showing competitive inhibition of CA-CB interaction by NBD1. When NBD1 was immobilized on the sensor chip, the CB subunit of crotoxin binds to NBD1 but the CA subunit does not interact with CB since the CA-binding site is occupied by NBD1. (B) The spectrofluorimetric assay showing the competitive inhibition of PLA₂ activity by oxidiazolone (PMS 1062). PLA₂ activity of CB was determined by spectrofluorimetric assay (Radvanyi, F., Jordan, L., Russo-Marie, F. and Bon, C. (1989) A sensitive and continuous fluorometric assay for phospholipase A2 using pyrene-labeled phospholipids in the presence of serum albumin. Analytical biochemistry, 177, 103-109) using β-py-C₁₀-PG as substrate. The effect of PMS 1062 on PLA₂ enzymatic activity of CB (B) and CB/NBD1 (C) was determined in the reaction mixture under standard conditions with substrate concentrations of 5 μM and 10 μM in the presence of 0-8 μM PMS 1062. Dixon plots show that a 20-fold higher concentration of the inhibitor is required for inhibition of 50% of PLA₂ activity in the complex demonstrating that the catalytic site of CB is masked by NBD1. (C) Comparison of CA and ΔF508-NBD1 binding sites of CBb, illustrating that the CA-CB and CB-/ΔF508-NBD1-binding sites significantly overlap. The SASA of CBb subunit is shown. The buried surface representing the common interface of CBb-CA₂ and CBb/ΔF508-NBD1 is shown in blue. Residues participating only in binding of ΔF508-NBD1 and CBb, are shown in pink and yellow, respectively.

FIG. 13 shows kinetic plots of selected peptides from the regions of interest in both WT (upper panels) and ΔF508 NBD1 (lower panels) showing gradual increase in deuterium uptake at different times of incubation in D2O (10 seconds, 1 minute and 20 minutes). The black lines link datapoints obtained for unbound NBDs and the green lines for NBD1-CB crotoxin subunit complex. Error bars represent the range between the data points measured in two independent experiments.

FIGS. 14A and 14B show HDX-MS analysis of NBD1-CA complex. Difference in the deuteration (%) before and after addition of CA crotoxin subunit in (A) WTNBD1 and (B) ΔF508NBD1 respectively after 10 s of exchange.

FIG. 15 shows configuration of the CBb unit (SEQ ID NO: 7).

FIG. 16 shows the topology of peptides NS-9 (105-113) with SEQ ID NO: 3, peptide NQ-18 (16-33) with SEQ ID NO: 2 and peptide HK-7 (1-7) with SEQ ID NO: 1 is represented in the CBb unit.

FIG. 17 shows the SPR binding of peptide NQ-18 (16-33) which interacts with ΔF508-NBD1 and increases Cl— channel current. Peptide concentration injected is 25-50-100 and 200 μM. The binding buffer is 4% ACM.

FIG. 18 shows alternative peptides in the CBb unit and their location. Peptide NG-13 (105-117) has the sequence if SEQ ID NO: 4, peptide NR-20 (16-35) has the sequence of SEQ ID NO: 5 and peptide HK-13 (1-13) has the sequence of SEQ ID NO: 6. FIG. 18 also discloses SEQ ID NO: 7.

### DETAILED DESCRIPTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. Certain references and other documents cited herein are expressly incorporated herein by reference. Additionally, all Genbank or other sequence database records cited herein are hereby incorporated herein by reference. In case of conflict, the present specification, including definitions, will control. The materials, methods, and examples are illustrative only and not intended to be limiting.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3d ed., Cold Spring Harbor Laboratory Press, Cold Spring Harbor, N.Y. (2001); Ausubel et al., Current Protocols in Molecular Biology, Greene Publishing Associates (1992, and Supplements to 2002); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

Before the present compositions, methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe.

As used herein, “subject” means any mammal including mice or primates. In a preferred embodiment the subject is a human.

As used herein, the terms “treat,” “treatment,” “treating,” and “amelioration” refer to therapeutic treatments, wherein the object is to reverse, alleviate, ameliorate, inhibit, slow down and/or stop the progression or severity of a condition associated with a disease or disorder. The terms include reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The terms “treat,” “treatment,” “treating,” and “amelioration” in reference to a disease also include providing relief from the symptoms or side-effects of the disease (including palliative treatment).

As used herein, an “effective amount” is an amount of a chemical entity that is effective when administered following a dosing schedule over a therapeutic dosing period.

As used herein, a “therapeutic dosing period” is a period of time during which a chemical entity is administered to a subject following a defined dosing schedule.

The human CFTR gene and protein are known in the art. The human CFTR sequence is available at www.uniprot.org/uniprot/P13569.

### A. Peptide Modulators

Peptide modulators of CFTR are provided. The peptide modulators bind to the nucleotide binding domain 1 (NBD1) of CFTR. In some embodiments binding of the peptide modulator to CFTR increases CFTR activity. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and by increasing the plasma membrane fraction of CFTR in a cell comprising the CFTR.

A template-based modelling approach was used to select peptides to effectively disrupt interactions between delF508-NBD1 and CB subunit of Crotoxin. As a starting point, a structural 3D model of the delF508NBD1/CBb complex were created. A molecular docking protocol consisting of the following steps was used: (a) an initial, rigid body 3D search based on fast a Fourier transform algorithm; (b) primary rescoring with a linear weighted scoring function implemented in ZRANK; (c) structural refinement by Monte Carlo methods; (d) secondary rescoring with ZRANK function optimized for refined complexes. The highest scored model of the delF508-NBD1/CB complex structure, has been used for identification of protein-protein interaction interface and then to proposed final peptide sequences on the basis of the native sequence of CB. This approach was used to identify the following peptides: HLLQFNK (SEQ ID NO: 1), NAVPFYAFYGCYCGWGGQ (SEQ ID NO: 2), NGYMFYPDS (SEQ ID NO: 3).

In some embodiments the peptide modulator comprises or consists of an amino acid fragment of the CB subunit of crotoxin from Crotalus durrissus terrificus venom. In some embodiments the peptide modulator comprises the amino acid sequence HLLQFNK (SEQ ID NO: 1) or a functional variant of SEQ ID NO: 1. In some embodiments the peptide modulator consists of the amino acid sequence SEQ ID NO: 1 or a functional variant of SEQ ID NO: 1.

In some embodiments the peptide modulator comprises the amino acid sequence NAVPFYAFYGCYCGWGGQ (SEQ ID NO: 2) or a functional variant of SEQ ID NO: 2. In some embodiments the peptide modulator consists of the amino acid sequence SEQ ID NO: 2 or a functional variant of SEQ ID NO: 2.

In some embodiments the peptide modulator comprises the amino acid sequence NGYMFYPDS (SEQ ID NO: 3) or a functional variant of SEQ ID NO: 3. In some embodiments the peptide modulator consists of the amino acid sequence SEQ ID NO: 3 or a functional variant of SEQ ID NO: 3.

In some embodiments the peptide modulator comprises the amino acid sequence NGYMFYPDSR CRG (SEQ ID NO: 4) or a functional variant of SEQ ID NO: 4. In some embodiments the peptide modulator consists of the amino acid sequence SEQ ID NO: 4 or a functional variant of SEQ ID NO: 4.

In some embodiments the peptide modulator comprises the amino acid sequence NAVPFYAFYG CYSGWGGQGR (SEQ ID NO: 5) or a functional variant of SEQ ID NO: 5. In some embodiments the peptide modulator consists of the amino acid sequence SEQ ID NO: 5 or a functional variant of SEQ ID NO: 5.

In some embodiments the peptide modulator comprises the amino acid sequence HLLQFNKMIK FET (SEQ ID NO: 6) or a functional variant of SEQ ID NO: 6. In some embodiments the peptide modulator consists of the amino acid sequence SEQ ID NO: 6 or a functional variant of SEQ ID NO: 6.

In some embodiments the peptide modulator is recombinant. In some embodiments the peptide modulator is synthetic. In some embodiments the peptide modulator is isolated. In some embodiments the peptide modulator is purified.

The properties of the peptide modulator can be readily verified by techniques known to those skilled in the art, such as those described in the examples of the present application.

“Functional” with respect to a peptide modulator refers to a peptide which is able to bind to CFTR protein and increase CFTR activity in a cell. A “functional variant” of an amino acid sequence is an amino acid sequence that has at least one sequence modification in comparison to a reference sequence; and that is able to bind to CFTR protein and increase CFTR activity in a cell. In some embodiments the functional variant increases CFTR activity in a cell by at least 25%, at least 50%, at least 75%, at least 80%, at least 85%, at least 90%, at least 95%, or at least 100% of the increase in CFTR activity in a cell that is achieved by a peptide modulator comprising the reference sequence. In some embodiments the functional variant increases CFTR activity in a cell by at least 25%, at least 50%, at least 75%, at least 80%, at least 85%, at least 90%, at least 95%, or at least 100% of the increase in CFTR activity in a cell that is achieved by the CB subunit of crotoxin from Crotalus durrissus terrificus venom. Suitable assays for making this comparison are provided, for example, in the examples of this application.

Functional variants may be derived from wild-type amino acid sequences by the introduction of one or more mutations (deletion, insertion, and/or substitution) at specific amino acid positions. In some embodiments the functional variant differs from the wild-type amino acid sequence by the deletion, insertion, and/or substitution of 1, 2, 3, 4, 5, 6, 7, 8, 9, or 10 or more amino acids. In some embodiments the functional variant comprises one or more deletion, and/or one or more insertion, and/or one or more substitution relative to the wild-type amino acid sequence. In a particular embodiment functional variants are obtained by insertion of amino acid residues at the N- or C-terminal end of the peptide. These variants may in particular result from addition of amino acid residues which are adjacent to those of the peptide in the CB unit in particular in the CBb sequence of SEQ ID NO: 7. Examples of such constructs are disclosed herein, in particular for peptides of less than 25 amino acid residus.

In some embodiments a functional variant comprises an amino acid sequence which is “substantially homologous” or “substantially similar” to the sequence of the reference peptide from which it is derived. Two amino acid sequences are “substantially homologous” or “substantially similar” when one or more amino acid residues are replaced by a biologically similar residue and/or when the sequences are at least 80% identical and/or at least 90% similar.

The percent amino acid sequence identity/similarity is defined as the percent of amino acid residues in a Compared Sequence that are identical/similar to the Reference Sequence after aligning the sequences and introducing gaps if necessary, to achieve the maximum sequence identity. The Percent identity is then determined according to the following formula: Percent identity=100×[1−(C/R)], wherein C is the number of differences between the Reference Sequence and the Compared sequence over the entire length of the Reference Sequence, wherein (i) each amino acid in the Reference Sequence that does not have a corresponding aligned amino acid in the Compared Sequence, (ii) each gap in the Reference Sequence, and (iii) each aligned amino acid in the Reference Sequence that is not identical/similar to an amino acid in the Compared Sequence constitutes a difference; and R is the number amino acids in the Reference Sequence over the length of the alignment with the Compared Sequence with any gap created in the Reference Sequence also being counted as an amino acid.

Alignment for purposes of determining percent amino acid sequence identity can be achieved in various ways known to a person of skill in the art, for instance using publicly available computer software such as BLAST (Altschul et al., J. Mol. Biol., 1990, 215, 403-), FASTA, the GCG (Genetics computer Group, Program Manual for the GCG Package, version 7, Madison, Wis.) pileup program, or any of the programs known in the art. When using such software, the default parameters, e.g., for gap penalty and extension penalty, are preferably used. For amino acid sequences, the BLASTP program uses as default a word length (W) of 3 and an expectation (E) of 10.

Conservative substitution refers to the substitution of one amino acid with another, without altering the overall conformation and function of the peptide, including but not limited to the replacement of an amino acid with one which has similar chemical or physical properties (size, charge or polarity), which generally does not modify the functional properties of the protein. Amino acids with similar properties are well known in the art. A non-limitative example of conservative substitution(s) comprises the five following groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W). Alternative, examples of conservative substitutions are known in the art.

In some embodiments the functional variant comprises or consists of an amino acid sequence which is at least 70%, 80%, 85%, 90% or 95% identical to SEQ ID NO: 1, 2 3, 4, 5 or 6. In some embodiments the functional variant differs from SEQ ID NO: 1, 2 or 3 by one or more conservative substitutions.

In some embodiments the peptide modulator comprises no more than 100, 90, 80, 70, 65, 60, 55, 50, 45, 40, 35, 30, 25, 20, 15, 10, 9, 8, or 7 amino acids. In a particular embodiment, the peptide modulator that comprises the amino acid sequence of SEQ ID NO: 1 is the peptide of amino acid sequence SEQ ID NO: 6 or a peptide which comprises the amino acid sequence SEQ ID NO: 6, especially having at most a number of amino acid residues as disclosed herein. In a particular embodiment, the peptide modulator that comprises the amino acid sequence of SEQ ID NO: 2 is the peptide of amino acid sequence SEQ ID NO: 5 or a peptide which comprises the amino acid sequence SEQ ID NO: 5, especially having at most a number of amino acid residues as disclosed herein. In a particular embodiment, the peptide modulator that comprises the amino acid sequence of SEQ ID NO: 3 is the peptide of amino acid sequence SEQ ID NO: 4 or a peptide which comprises the amino acid sequence SEQ ID NO: 4, especially having at most a number of amino acid residues as disclosed herein. In a particular embodiment the peptide modulator consists in a sequence of 7 to 30 or 7 to 25 amino acid residues.

In some embodiments the peptide modulator comprises a first amino acid sequence selected from SEQ ID NOS: 1-6 or in particular SEQ ID NOS: 1-3 or 4-6, or a functional variant thereof, fused to a second amino acid sequence. The second amino acid sequence may be fused to the N-terminal and/or C-terminal end(s) of the amino acid sequence selected from SEQ ID NOS: 1-6 or in particular SEQ ID NOS: 1-3 or 4-6. The second amino acid sequence may be selected to facilitate the purification, detection, immobilization, and/or cellular targeting of the peptide modulator, and/or to increase the affinity of the peptide modulator for CFTR, the bioavailability of the peptide modulator, the production in expression systems of the peptide modulator and/or the stability of the peptide modulator. The second amino acid sequence may be selected from: (i) a cell-penetrating moiety, (ii) a labeling moiety such as a fluorescent protein (GFP and its derivatives, BFP and YFP), (iii) a reporter moiety such as an enzyme tag (luciferase, alkaline phosphatase, glutathione-S-transferase (GST), β-galactosidase), (iv) a binding moiety such as an epitope tag (polyHis6 (SEQ ID NO: 8), FLAG, HA, myc.), a DNA-binding domain, a hormone-binding domain, a poly-lysine tag for immobilization onto a support, (v) a stabilization moiety, and (vi) a targeting moiety for addressing the peptide modulator to a specific cell type or subcellular compartment. In addition, the amino acid sequence selected from SEQ ID NOS: 1-6, or a functional derivative thereof, may be separated from the second amino acid sequence by a linker which is long enough to avoid inhibiting interactions between the amino acid sequence selected from SEQ ID NOS: 1-6, or a functional derivative thereof, and the second amino acid sequence. The linker may comprise a recognition site for a protease, for example, for removing affinity tags and/or stabilization moieties.

In some embodiments the second amino acid sequence is a cell-penetrating peptide (CPP), also known as protein transduction domains (PTDs), membrane translocation sequences (MTSs), transport peptides, carrier peptides or Trojan peptides are well-known in the art. In some embodiments, the CPP aids translocation of the peptide modulator into cells at significantly higher levels than passive diffusion, without causing substantial membrane damage, and can be used as vectors of other molecules when linked to them.

In some embodiments the peptide modulator comprises a chemical modification. In some embodiments all or substantially all of the amino acids of a peptide modulator comprise a similar or identical chemical modification. In some embodiments a subset of at least one of the amino acids of a peptide modulator comprise a similar or identical chemical modification. In some embodiments the chemical modification comprises at lease one of: the substitution of a natural amino acid with a non-proteinogenic amino acid (D amino acid or amino acid analog); the modification of the peptide bond, in particular with a bond of the retro or retro-inverso type or a bond different from the peptide bond; the cyclization, and the addition of a chemical group to the side chain or the end(s) of the peptide, in particular for coupling an agent of interest. These modifications may be used to label the peptide, and/or to increase its stability and/or its resistance to proteolysis.

In some embodiments the at least one chemical modification protects the peptide modulator against proteolysis.

In some embodiments the N- and/or C-terminus of the peptide modulator is protected against proteolysis.

In some embodiments the N-terminus is in the form of an acetyl group and/or the C-terminus in the form of an amide group.

In some embodiments the peptide modulator is protected against proteolysis by internal modifications such as the replacement of at least one —CONH— peptide bond by a (CH2NH) reduced bond, a (NHCO) retro-inverso bond, a (CH2-O) methylene-oxy bond, a (CH2-S) thiomethylene bond, a (CH2CH2) carba bond, a (CO—CH) cetomethylene bond, a (CHOH—CH2) hydroxyethylene bond, a (N—N) bond, a E-alcene bond, or a —CH═CH— bond.

In some embodiments the peptide modulator is modified by at least one of acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

In some embodiments the peptide modulator comprises at least one amino acid in D configuration.

In some embodiments the peptide modulator is stabilised by intramolecular crosslinking, by modifying at least two amino acid residues with olefinic side chains, preferably C3-C8 alkenyl chains, more preferably penten-2-yl chains, followed by crossliking of the chains according to the so-called ‘“stapled-peptide technology” described in Walensky et al., Science, 2004, 305, 1466-1470.

In some embodiments the peptide modulator is stabilised by covalent binding to a polyethylene glycol (PEG) molecule, preferably a PEG of 1500 Da or 4000 Da, advantageously bound to their C-terminus or a lysine residue. Such coupling has the advantage to decrease urinary clearance and therapeutic doses and increase half-life in blood plasma.

In some embodiments the peptide modulator is stabilised and its half-life increased by incorporation into a biodegradable and biocompatible polymer material for drug delivery system forming microspheres, such as for instance poly(D, L-lactide-co-glycolide (PLGA) and nanoparticules.

### B. Pharmaceutical Compositions

Also provided are pharmaceutical compositions comprising a peptide modulator of this disclosure. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3; a polypeptide comprising the amino acid sequence NGYMFYPDSR CRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYG CYSGWGGQGR (SEQ ID NO: 5), a polypeptide consisting of the amino acid sequence SEQ ID NO: 5; and a polypeptide comprising a functional variant of SEQ ID NO: 5; a polypeptide comprising the amino acid sequence HLLQFNKMIK FET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

Typically a pharmaceutical composition comprises a peptide modulator as described above, and a pharmaceutically acceptable carrier. In some embodiments the pharmaceutical composition is formulated for administration by a route selected from oral, parenteral and local.

The pharmaceutical composition comprises a therapeutically effective amount of the peptide modulator, e.g., sufficient to show benefit to the subject to whom it is administered. The pharmaceutically effective dose depends upon the composition used, the route of administration, the type of subject being treated, the physical characteristics of the subject, concurrent medication, cystic fibrosis disease state and other factors, that those skilled in the art will recognize.

### C. Methods of Increasing CFTR Activity in a Cell

Methods of increasing CFTR activity in a cell are also provided. The methods comprise contacting the cell with a peptide modulator of CFTR to thereby increase CFTR activity in the cell. In some embodiments the peptide modulator binds to the nucleotide binding domain 1 (NBD1) of CFTR. In some embodiments the peptide modulator to CFTR increases CFTR activity by increasing Cl⁻ channel current in a cell comprising the CFTR. In some embodiments the peptide modulator to CFTR increases CFTR activity by increasing the plasma membrane fraction of CFTR in a cell comprising the CFTR. In some embodiments the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and a polypeptide comprising the amino acid sequence NGYMFYPDS (SEQ ID NO: 3), a polypeptide consisting of the amino acid sequence SEQ ID NO: 3, a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

The cell may be contacted with the peptide modulator using any technique known in the art. In general the peptide modulator will be provided to the cell in a form and/or using a method such that at least some of the peptide modulator enters the cell and becomes intracellular. In some embodiments this is achieved by incorporating a polypeptide sequence that ius a cell penetrating peptide into the peptide modulator. In some embodiments this is achieved by introducing a nucleic acid sequence into the cell that encodes the peptide modulator under conditions such that the peptide modulator is synthesized intracellularly to thereby provide the peptide modulator to the cell.

### D. Methods of Treating Cystic Fibrosis in a Subject

Also provided are methods of treating cystic fibrosis in a subject in need thereof. In some embodiments the methods comprise administering an effective amount of a peptide modulator of CFTR to the subject to thereby increase CFTR activity in the subject. In some embodiments the peptide modulator binds to the nucleotide binding domain 1 (NBD1) of CFTR. In some embodiments the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, a polypeptide comprising a functional variant of SEQ ID NO: 2; and a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

Typically an effective amount of the peptide modulator is administered to the subject for a therapeutic dosing period. The therapeutic dosing period is chosen to allow improvement in at least one symptom or feature of cystic fibrosis in a subject. In some embodiments the at least one feature is use of a concurrent medication and improvement is a reduction in the amount and/or frequency of administration of a second cystic fibrosis therapy. In some embodiments the second cystic fibrosis therapy is an antibiotic. In some embodiments the second cystic fibrosis therapy is a mechanical lung treatment or therapy.

In some embodiments the subject is a human.

In some embodiments the subject is heterozygous for the ΔF508CFTR allele. In some embodiments the subject is homozygous for the ΔF508CFTR allele. In some embodiments the subject does not comprise a ΔF508CFTR allele.

### E. Uses

Also provided are uses of a peptide modulator of CFTR for the manufacture of a medicament for use in treating cystic fibrosis. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; a polypeptide comprising the amino acid sequence NGYMFYPDS (SEQ ID NO: 3), a polypeptide consisting of the amino acid sequence SEQ ID NO: 3, a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

Also provided are peptide modulators of CFTR for use in treating cystic fibrosis in a subject. In some embodiments the peptide modulator binds to the nucleotide binding domain 1 (NBD1) of CFTR. In some embodiments binding of the peptide modulator to CFTR increases CFTR activity by increasing Cl⁻ channel current in a cell comprising the CFTR. In some embodiments binding of the peptide modulator to CFTR increases CFTR activity by increasing the plasma membrane fraction of CFTR in a cell comprising the CFTR. In some embodiments binding of the peptide modulator to CFTR increases CFTR activity by increasing Cl⁻ channel current in a cell comprising the CFTR and increasing the plasma membrane fraction of CFTR in a cell comprising the CFTR. In some embodiments the CFTR is ΔF508CFTR. In some embodiments the peptide modulator comprises or consists of an amino acid fragment of the CB subunit of crotoxin from Crotalus durrissus terrificus venom. In some embodiments the peptide modulator is selected from: a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1; a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; a polypeptide comprising the amino acid sequence NGYMFYPDS (SEQ ID NO: 3), a polypeptide consisting of the amino acid sequence SEQ ID NO: 3, a polypeptide comprising a functional variant of SEQ ID NO: 3; a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4; a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6. In some embodiments the peptide modulator comprises a chemical modification.

### EXAMPLES

### Example 1: Materials and Methods

Biological Material:

Snake venom PLA₂s (heterodimeric crotoxin [CACB complex], its nonenzymatic CA subunit and three isoforms of the CB subunit [CBa₂, CBb and CBc] were purified as previously described¹⁶; recombinant human PLA₂ was produced as previously described²⁸ and stored at −20° C. All chemicals were purchased from Sigma. Anti-CFTR antibodies MM13-R were from EMD Millipore (Ma, USA). CFTR correctors, Corr4a and VX-809 were kindly provided by Cystic Fibrosis Foundation Therapeutics. CFTR inhibtor glyH101 was from EMD Millipore (Ma, USA) and Inh172 was from Calbiochem. Glibenclamide, amiloride, bumetanide were from Sigma-Aldrich.

Preparation of Oocytes:

After the partial ovariectomy of an anesthetized animal, oocytes were defolliculated by gentle shaking in calcium-free ND96 solution containing 96 mM NaCl, 2 mM KCl, 1 mM MgCl₂, 5 mM HEPES pH 7.5 supplemented with 0.4 U/ml collagenase (Type lA, Sigma). Healthy stage V-VI oocytes were selected for experiments. All animal protocols were approved by the Necker Faculty of Medicine Animal Care and Use Committee (University of Paris Descartes); authorization no. 7514, Ministry of Agriculture and Fishery and conformed to European Community regulations for the use of animals in research (authorization no. P2.AE.092.09). Animals (male and females) used in this study were from FVB/N strain. Mice were obtained from CDTA (Orleans, France, provided by Erasmus University, Rotterdam, Netherland) and housed at the SPF Animal Care Facility of Necker Faculty of Medicine.

Cloning:

Human CFTR cDNA was subcloned into pT7TS plasmid flanked by the 3′ and 5′ untranslated regions of the Xenopus β-globin gene. The cDNA coding for ΔF508CFTR was obtained from the wild-type construct using the Quick Change Site-Directed Mutagenesis Kit (Stratagene). Capped cRNAs were synthesized in vitro from the constructs, linearized by Sma I, using Riprobe in Vitro Transcription System Kit (mMESSAGE mMACHINE, Ambion). Stage V-VI oocytes were injected (Inject+Matic microinjector, Geneva, Switzerland) with 1 ng of CFTR cRNA or ΔF508-CFTR cRNA solubilised in 50 nl water, or with water alone (control oocytes)^{29; 30} and incubated at 18° C. in ND96 supplemented with penicillin-streptomycin for 3-4 days before further experiments.

Two-electrode voltage-clamp experiments (TEVC): Oocytes were placed in a microchamber and punctured with two low-resistance (0.5-3 Mohm), 3M KCl-filled microelectrodes. To reduce series resistance-induced errors during voltage clamp measurements, a virtual ground amplifier (VG2-A 100, Axon Inst, Union City, Calif., USA) was connected to the current-voltage amplifier (Axoclamp 2B, Union City, Calif., USA) and to bath electrodes (an agar-3M KCl bridge electrode and an Ag—AgCl pellet)³¹. Current voltage (I/V) relationships were obtained by applying from the resting membrane potential+20 mV voltage steps using Clampex9-generated protocol (Axon Instruments). Results were analysed using the program P-Clamp 9 (Axon Instruments). During the experiements, oocytes were superfused with ND96 Ringer's solution or with solutions differing from each other by a single parameter. Solution changes were commanded electronically using a laboratory-made device. Protein Kinase A (PKA) stimulation was achieved using stimulating cocktail consisting of ND96 supplemented with forskolin (Fsk, 1 μM) and IsoButylMethylXanthine (IBMX, 100 μM). CFTR-induced currents was taken as the difference in whole cell currents measured before and after exposure to the PKA-stimulating cocktail solution and the selective CFTR blocker (Inh172)³² obtained from Calbiochem. Other chemicals were purchased from Sigma. On the day of the electrophysiological experiment, the various PLA₂ were injected into oocytes (CFTR- or DF508-CFTR-expressing oocytes, or control oocytes). Crotoxin (CACB complex, CA, and CB subunits) and human sPLA₂ were solubilized in buffer mimicking an intracellular solution: potassium Glutamate 128 mM, NaCl 5 mM, MgSO₄ 7 mM, Hepes/KOH 20 mM, pH 7.0)³⁰ at protein concentrations of 50 μg/ml, 10 μg/ml and 12.5 μg/ml, respectively. After injecting 50 nl of the PLA₂-containing buffer, TEVC experiments were performed as described above. CFTR-mediated currents were measured before and after injection of PLA₂ using the same oocyte.

Short-Circuit Current Measurements:

Mice were 32 to 38 weeks old and weighed 23 to 36 g. Pups were weaned at 21 days and fed solid mouse chow (Teklad 2018S, Harland). All mice were allowed food and water ad libitum until the time of sacrifice by cervical dislocation. Colons were dissected, cut in 4 pieces from rectum to lower intestine. The fragments from colon were introduced immediately into Ussing chambers for subsequent short-circuit experiments.

Ussing Chamber:

Transepithelial transport measurements were performed as described previously³³ in the short-circuit mode (6 mice and 17 tissus). Mice had a cervical dislocation before excising intestine for isolated tissue preparations. The excised colon were stripped of external muscle layers and mounted in Ussing chambers (0.018 cm² aperture). Hemichambers were connected to a DVC-1000 voltage clamp (World Precision Instruments, Inc., Sarasota, Fla.) via Ag/AgCl electrodes and 3 M KCl agar bridges for recording of short-circuit current. Currents were stored on a computer using analog-to-digital converter (PowerLab) and LabChart software 5.0. Prior to the experiment, prostaglandin generation was blocked with 10 μM indomethacin on the apical and basolateral sides (incubation of tissue for 30 min before the beginning of the experiment). Transepithelial Isc was calculated as μEq cm⁻² tissue surface area. The apical and basolateral solutions contained (in mM): 116 NaCl, 25 NaHCO₃, 1.2 CaCl₂, 1.2 MgCl₂, 2,4 H₂HPO₄, 0,4 KH₂PO₄, 10 glucose and was gassed with 95% O₂-5% CO₂ (pH 7.4).

Patch-Clamp Experiments on HeLa Cells:

HeLa cells stably transfected with WT-CFTR were kindly provided by Pascale Fanen (INSERM U955, Créteil, France). The intracellular side of the membrane patch was exposed to a solution containing (in mM): 150 N-methyl-D-gluconate chloride (NMDG-Cl), 0.5 MgCl₂, 2 EGTA, 10 HEPES, 10 glucose, pH 7.4, adjusted to pH 7.4 with NMDG. The patch pipettes were filled with (in mM): 150 NMDG-Cl, 0.5 MgCl₂, 2 EGTA, 10 HEPES, 10 glucose, 1 ATP-Mg adjusted to pH 7.4 with NMDG. CFTR channels were activated by addition of 40 nM PKA+1 mM ATP-Mg at the intracellular side of the patches. The presence of CFTR channels was verified by addition of 100 μM glibenclamide and 10 μM inh172. The effect of CB was tested by perfusion of 1 nM of CB in the intracellular solution in the presence of 40 nM PKA+0.05, 0.5, or 1 mM ATP-Mg. The currents were recorded as previously described³⁴. Briefly, patch-clamp pipettes were pulled in two stages with a Kopf puller (Tujunga, Calif., USA) using borosilicate glass (GC150T, Harvard Apparatus, Edenbridge, Kent, UK). They were coated with Sylgard and polished just before use. Currents were recorded with Bio-logic RK 400 patch-clamp amplifiers (Bio-Logic, Claix, France), monitored using PClamp9 software (Axon Instruments, Foster City, Calif., USA) and stored on a computer. The experiments were carried out at room temperature (22-27° C.). For analysis of channel activity, current recordings were filtered at 300 Hz low-pass using an 8-pole Bessel filter (LPBF-48DG, NPI Electronic, Tamm, Germany) and digitized at a sampling rate of 1-2 kHz using a Digidata 1200 analog-to-digital converter and PClamp9 software (Axon Instruments, Foster City, Calif., USA). We calculated channel activity (NPₒ) as <I>/i, where <I> is the time-averaged current passing through the channels on the patch with the closed current level as reference and i is the unit current amplitude.

Whole-Cell Patch-Clamp Recordings:

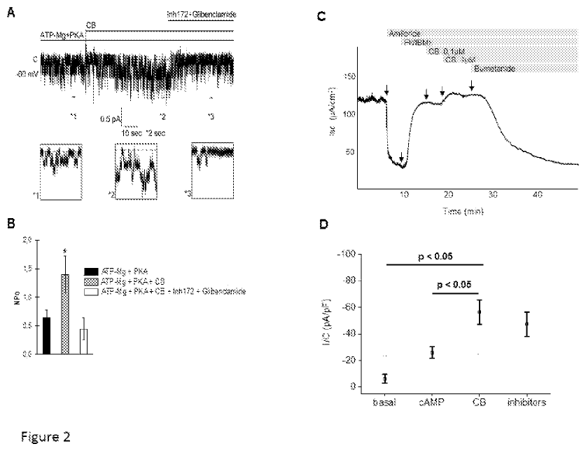

Nystatin-perforated whole-cell patch-clamp experiments were performed as previously described⁹. Detailed information is provided in the legend of FIG. 2D. The nystatin stock solution (50 mg/ml) was prepared daily in DMSO. Aliquotes were diluted (1:250) with an intermediate solution containing (in mM): 131 NaCl, 2 MgCl₂ and 10 Hepes, pH 7.3 (adjusted with NaOH), and sonicated for 1 min. The bath solution contained (in mM): 150 NaCl, 1 CaCl₂, 1 MgCl₂, 35 sucrose and 10 Hepes-Na⁺, pH 7.3 (adjusted with NaOH). Currents were recorded by application of regular pulses of −60 mV for is, with a holding potential of 0 mV and an interval of 3 s. To establish the I-V curves, regular voltage pulses were interrupted by a series of 9 voltage jumps (1-s duration each) toward membrane potentials between −100 and +80 mV. CFTR Cl⁻ currents, I_{CFTR}, were activated using 400 μM 8-(4-chlorophenylthio)-cAMP sodium salt (CPT-cAMP) and 100 μM 3-isobutyl-1-methylxanthine (IBMX). When maximal stimulation was reached, cells were bathed with 1 nM CB in the presence of CPT-cAMP and IBMX and 5 μM of the CFTR inhibitor, (CFTRᵢₙₕ172+GlyH101) or 100 μM glibenclamide, was added to the CPT-cAMP containing perfusion solution (solution +/−CB). I_{CFTR}, defined CFTR currents as a difference in current amplitude recorded during maximum stimulation with solution +/−CB and maximum inhibition with CFTRᵢₙₕ172. Each recording was performed after 5 to 7 minutes of stimulation or inhibition.

Analysis of protein expression by Western blot: After being washed three times in cold PBS buffer, oocytes were lysed in an ice cold solution containing 250 mM sucrose, 0.5 mM EDTA, 5 mM Tris-HCl, pH 7.4. supplemented with a protease inhibitor cocktail solution (Complete Mini, Roche, Indianapolis). For microsmal preparations enriched in plasma membranes, cell lysates were centrifuged at 200, 400 and 800 g (10 min each, 4° C.). The final supernatant was centrifuged at 100 000 g (1 hour, 4° C.). The resulting pellet, containing total membrane proteins, was resuspended in the lysis buffer. Equal protein samples were separated by SDS-8% PAGE and subjected to Western blotting. The blots were incubated with anti-CFTR MM13.4 monoclonal antibody (Millipore, Ma, USA) 1000-fold diluted. This antibody recognizes fully glycosylated CFTR (band C, 170-190 kDa), and immature CFTR (band B, 150-155 kDa). Anti mouse IgG, 5000-fold diluted (GE Healthcare) coupled to horseradish peroxidase was used as secondary antibody. Stained proteins were detected using an enhanced chemoluminescence ECL system (GE Healthcare).

Surface Plasmon Resonance (SPR) Analysis:

Protein-protein interactions were studied in real time using a SPR Biacore 2000 system and CM5 sensor chips (Biacore AB, GE Healthcare). NBD1 (WT or mutant) was purified as previously described by³⁵. It was covalently immobilized at 15° C. via primary amino groups on the sensor chip surface as follows: the carboxymethylated dextran matrix was activated with 35 μl of EDC/NHS (1/1) mixture, 10 μl of NBD1 at a concentration of 50 μg/ml in 10 mM sodium acetate, pH 5.0, was injected and unreacted groups were blocked with 35 μl of ethanolamine (pH 8.5). A separate flow channel on the same sensor chip, reserved for control runs, was subjected to a blank immobilization run by preparing it in the same way but without NBD1. The running and dilution buffer had the following composition: 50 mM Tris, 150 mM NaCl, 5 mM MgCl₂ (pH 7.6), 0.005% P20, 1 mM DTT. The interaction between different sPLA₂ (crotoxin, CA, CB, hGIIA, GIB PLA₂s) and the immobilized NBD1 was monitored at 20 or 25° C. by injecting different concentrations (20, 10, 5, 2.5, 1.25 μg/ml) of the purified proteins, with a flow rate of 30 μl/min, and recording the refractive index changes at the sensor surface. The subsequent dissociation phase was followed after each association run by injecting the running buffer alone. Surfaces were regenerated by three washes with 20 μl of 5 mM NaOH followed by two washes with 20 μl of 1M NaCl. All association and dissociation curves were corrected for non-specific binding by subtraction of control curves obtained from injection of the analyte concentrations through the blank flow channel. The kinetic constants, kₒₙ and k_{off}, were calculated using Biacore BIAEVALUATION 4.1 software (Biacore AB, GE Healthcare), assuming a simple two-component model of interaction. Each run consisted of three independent measurements (three different immobilization flowpaths and one control flowpath).

Effect of Inhibitor PMS 1062 on the Interaction of CBa₂ with hNBD1:

PLA₂ activity was measured by fluorometric assay³⁶ using 1-hexadecanoyl-2-(1-pyrenyldecanoyl)-sn-glycero-3-phosphoglycerol ammonium salt (β-py-C₁₀-PG) (Molecular probes USA) as substrate. The effect of PMS 1062²² on inhibition of PLA₂ activity of CBd and the CBd/NBD1 complex was determined in reaction mixtures containing 50 mM TrisHCl pH 7.5, 0.05M NaCl, 1 mM EGTA, 0.1% BSA and 10 mM CaCl₂, with β-py-C₁₀-PG concentrations of 5.0 and 10.0 μM in the presence of 0-8.0 μM concentrations of inhibitor. The inhibition type of the enzymatic reaction was determined by graphical analysis using the Dixon method³⁷ and the inhibition constant (Kᵢ) values were calculated by Dixon plot.

Molecular Modeling of the ΔF508-NBD1/CBb Complex:

For molecular docking studies, crystal structures of ΔF508-NBD1 (1XMJ)³⁸ and crotoxin (3ROL)²⁴ were obtained from PDB³⁹. To study the interactions between F508-NBD1 and CBb, we applied a previously described procedure²⁵ slightly modified. Briefly, the initial stage docking algorithm of ZDOCK 3.0.2⁴⁰ was used with rotational sampling density set to 60. Fifty four thousand complexes generated by ZDOCK were rescored and reranked according to optimized energy-based function for initial-stage docking implemented in ZRANK⁴¹. Prior to rescoring, polar hydrogen atoms were added to the initial structures of both binding partners using SYBYLX2.0 (Tripos International). For each of the top 20 models proposed by ZDOCK/ZRANK combination, 300 refined structures were generated by the Monte Carlo (MC) refinement method of RosettaDock implemented in Rosetta v. 3.4 package⁴². This step was performed with extra chi1 and chi2-aromatic rotamers and with MC rigid-body perturbation parameters set to 4A and 0.20. Each of the 20 sets of 300 refined models was further treated separately. All complexes within the groups were rescored with the ZRANK scoring function optimized for refinement complexes. For each set, only the top scored model was selected, resulting in twenty new refined docking models, which were ordered according to the ZRANK score. Subsequently, all complexes were superimposed onto the structural model proposed by Serohijos⁴³. Structures in which CBb occupies the ΔF508-NBD1 inter-domain interfaces of CFTR were discarded. The final top structure among the remaining complexes was analyzed by Protein Interaction Calculator⁴⁴. All figures of the complex model were generated with the PyMOL Molecular Graphics System Version 1.5.0.4⁴⁵.

Hydrogen Deuterium Exchange (HDX):

The HDX-MS experiments were performed essentially as described in (doi: 10.1016/j.jsb.2015.10.001). In the first step of the analysis, the list of peptic peptides for both NBDs were established using a non-deuterated sample. An aliquot (5 μL) of the protein stock solution was diluted 10 times by adding to 45 μL of H₂O Reaction Buffers at room temperature (50 mM Tris-HCl, 150 mM NaCl, pH 7.6, 25° C.). The sample was then acidified by mixing with 10 μL of H₂O Stop Buffer (2 M Glycine buffer, pH 2.5). In the case of the NBD-CB complex, the samples were first mixed in 1:1 ratio before the HDX experiments were carried out. The sample was digested online using a 2.1 mm×30 mm immobilized pepsin resin column (Porozyme, ABI, Foster City, Calif.) with 0.07% formic acid in water as the mobile phase (200 μL/min flow rate). Peptic peptides were passed directly to the 2.1 mm×5 mm C18 trapping column (ACQUITY BEH C18 VanGuard precolumn, 1.7 μm resin, Waters, Milford, Mass.). Trapped peptides were eluted onto a reversed phase column (Acquity UPLC BEH C18 column, 1.0×100 mm, 1.7 m resin, Waters, Milford, Mass.) using a 6-40% gradient of acetonitrile in 0.1% formic acid at 40 μL/min, controlled by the nanoACQUITY Binary Solvent Manager. Total time of a single run was 13.5 minutes. All fluidics, valves, and columns were maintained at 0.5° C. using the HDX Manager (Waters, Milford, Mass.), with the exception of the pepsin digestion column which was kept at 20° C. inside the temperature controlled digestion column compartment of the HDX manager. The C18 column outlet was coupled directly to the ion source of SYNAPT G2 HDMS mass spectrometer (Waters, Milford, Mass.) working in Ion Mobility mode. Lock mass was activated and carried out using Leucine-enkephalin (Sigma). For protein identification, mass spectra were acquired in MS^{E} mode over the m/z range of 50-2000. The spectrometer parameters were as follows: ESI positive mode, capillary voltage 3 kV, sampling cone voltage 35 V, extraction cone voltage 3 V, the source temperature 80° C., desolvation temperature 175° C. and desolvation gas flow 800 L/h. The spectrometer was calibrated using standard calibrating solutions. Peptides were identified using ProteinLynx Global Server software (Waters, Milford, Mass.). The list of identified peptides containing peptide m/z, charge, retention time and ion mobility drift time was passed to the DynamX 2.0 hydrogen-deuterium data analysis program (Waters, Milford, Mass.). HDX experiments were carried out as described for the non-deuterated samples, with the Reaction Buffer prepared using D₂O (99.8% Cambridge Isotope Laboratories, Inc.) and pH (uncorrected meter reading) adjusted using DCl (Sigma). After mixing 5 μL protein stock with 45 μL D₂O Reaction buffer, the exchange reactions were carried out for varied time periods as specified in the text, at room temperature. The exchange was quenched by reducing the pH to 2.5 by adding the reaction mixture to Stop Buffer (2 M Glycine buffer, pH 2.5) and cooling on ice. Immediately after quenching in the Stop Buffer, the sample was manually injected into the nanoACQUITY (Waters, Milford, Mass.) UPLC system. Subsequently, pepsin digestion and LC and MS analyses were carried out exactly as described above for non-deuterated samples. Two control experiments were carried out to account for in- and out-exchange artifacts, as described previously (doi: 10.1016/j.jsb.2015.10.001). In brief, to assess minimum exchange (in-exchange control), D₂O Reaction Buffer was added to Stop Buffer that had been cooled on ice prior to addition of protein stock, and this mixture was immediately subjected to pepsin digestion and LC-MS analysis as described above. The deuteration level in an in-exchange experiment was calculated as described below and denoted as 0% exchange (Mₑₓ⁰). For out-exchange analysis, 5 μL of protein stock was mixed with 45 μL of D2O Reaction Buffer, incubated for 24 h, mixed with Stop Buffer, and analyzed as described above. The deuteration level in an out-exchange experiment was calculated and denoted as 100% exchange (Mₑₓ¹⁰⁰).

The above experimental scheme enabled us to obtain the same set of fragments from the control and HD exchange experiments. Each experiment was repeated twice and the results represent the mean of these replicates. The Y-error bars represent the range between the douplicate data points (doi:10.1021/bi3008998).

HDX data analysis: The deuteration level for each peptide resulting from exchange was calculated in an automated way using DynamX 2.0 software, based on the peptic peptide list obtained from the PLGS program, and further filtered by the DynamX 2.0 program with the following acceptance criteria: minimum intensity threshold −3000, minimum products per amino acids −0.3. The analysis of the isotopic envelopes after exchange was carried out by the DynamX 2.0 program with the following parameters: RT deviation+15 s, m/z deviation+12.5 ppm, drift time deviation+2 time bins. The average masses of peptides in the exchange experiment (Mex) and the two control experiments (Mex0 and Mex100) obtained from the automated analysis were then verified by visual inspection. Ambiguous or overlapping isotopic envelopes were discarded from further analysis. Whenever a split isotopic envelope was observed, the separate Mex values corresponding to each envelope were calculated using the MassLynx program. Final data were exported to Excel (Microsoft) spreadsheets for calculation of HD exchange mass shifts and fraction of exchange calculations. The percentage of relative deuterium uptake (% Deuteration) of a given peptide was calculated by taking into account both control values, following the formula: % Deuteration=((Mₑₓ−Mₑₓ⁰)/(Mₑₓ¹⁰⁰−Mₑₓ⁰))*100. Error bars for fraction of exchange were calculated for two independent experiments. The differences in exchange (ΔHDX−% Difference in deuteration) were obtained by subtracting the fraction of exchange under different conditions.

Statistics:

Except when stated, results were expressed as means±SEM, with n as the number of oocytes. Significance of the results was assessed by paired Student t-test using SigmaPlot (Systat software Inc., San Jose, Calif.). The difference was considered significant for P values <0.05.

### Example 2: Direct and Specific Binding of the CA-CB Complex and PLA₂ CB Subunit to Human NBD1 of CFTR

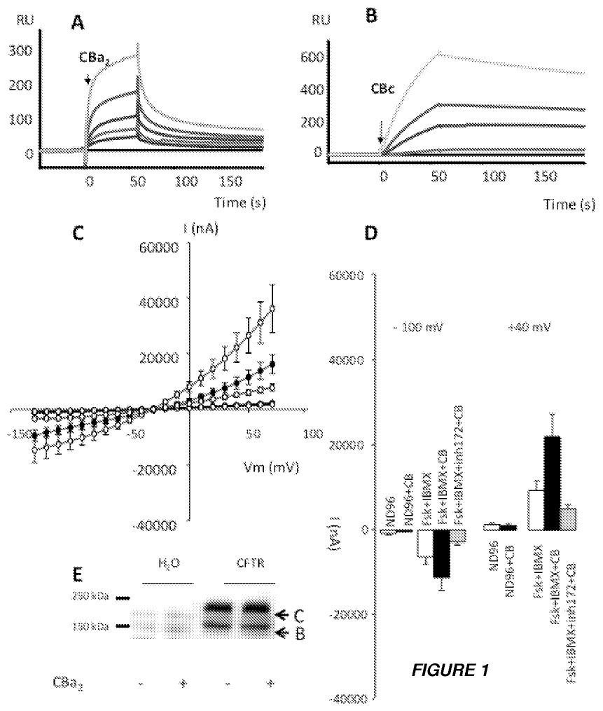

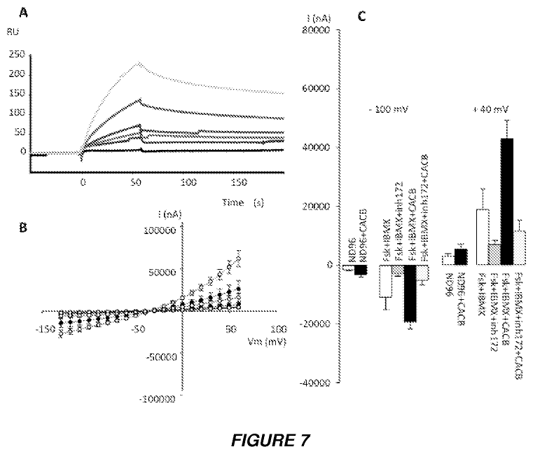

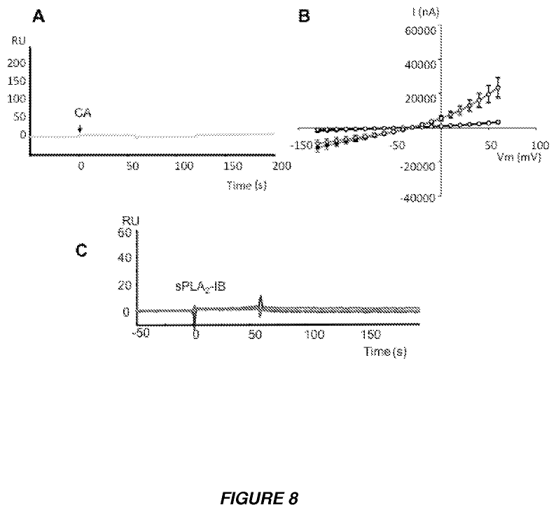

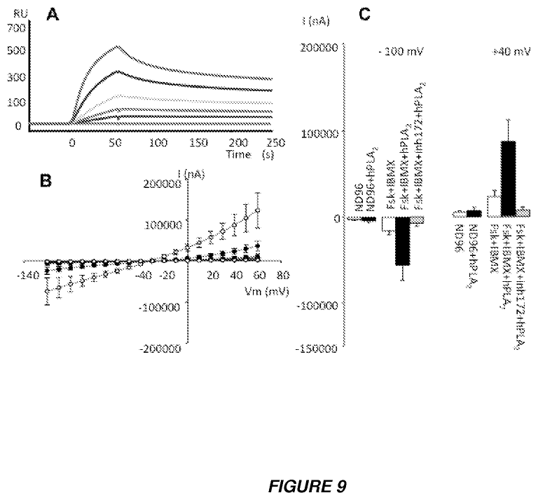

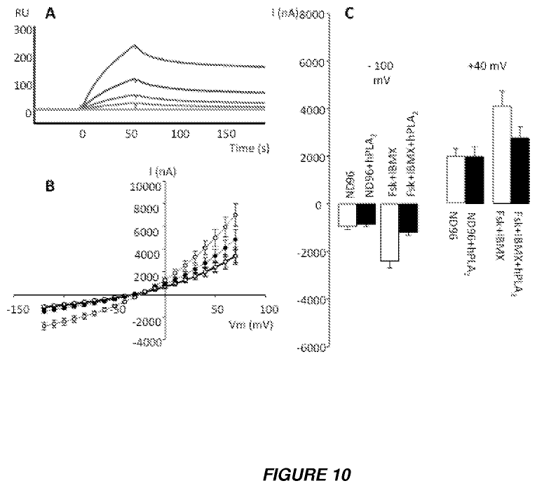

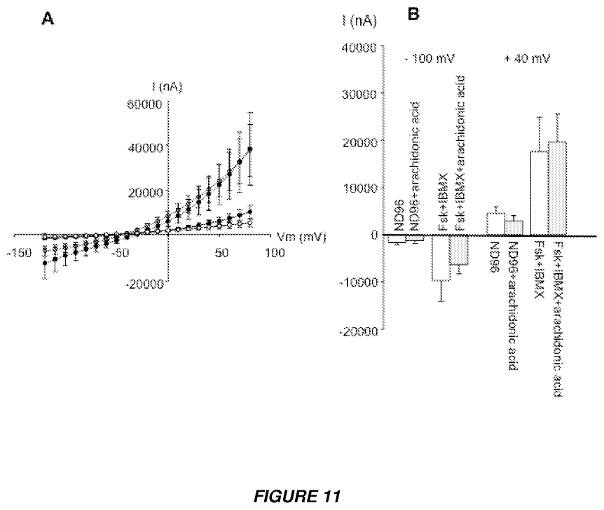

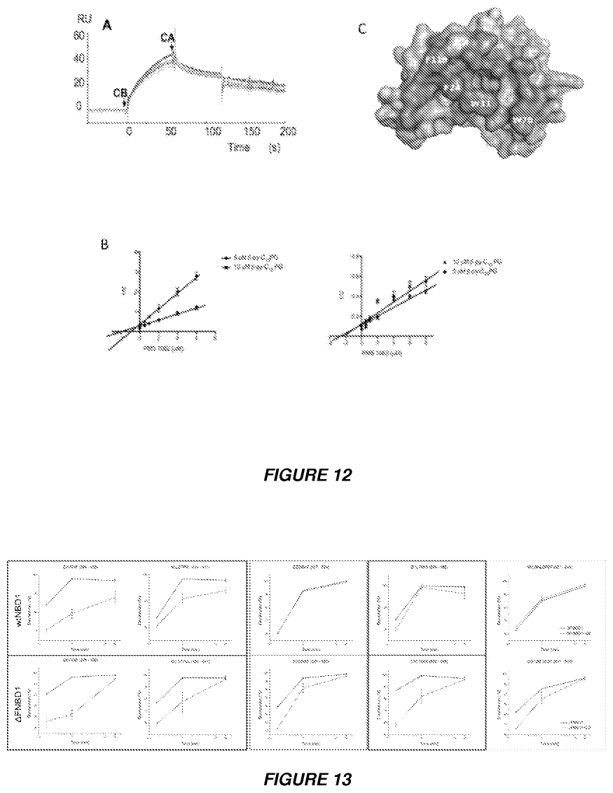

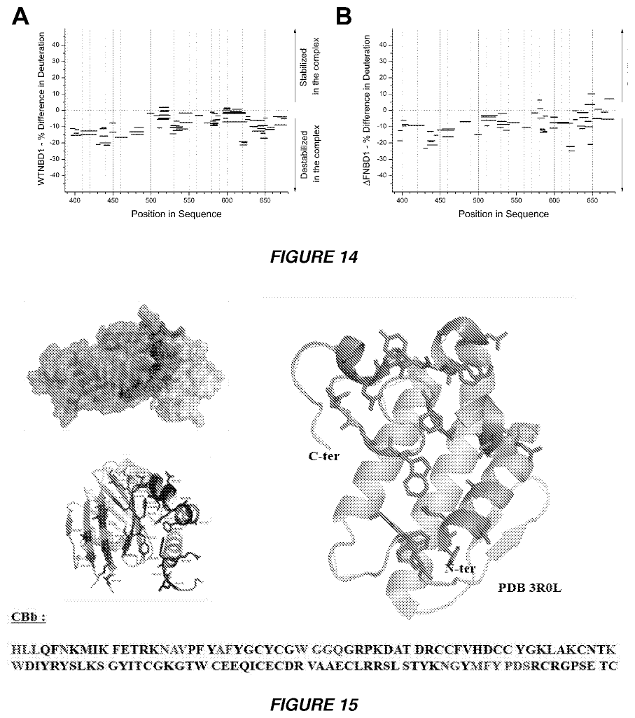

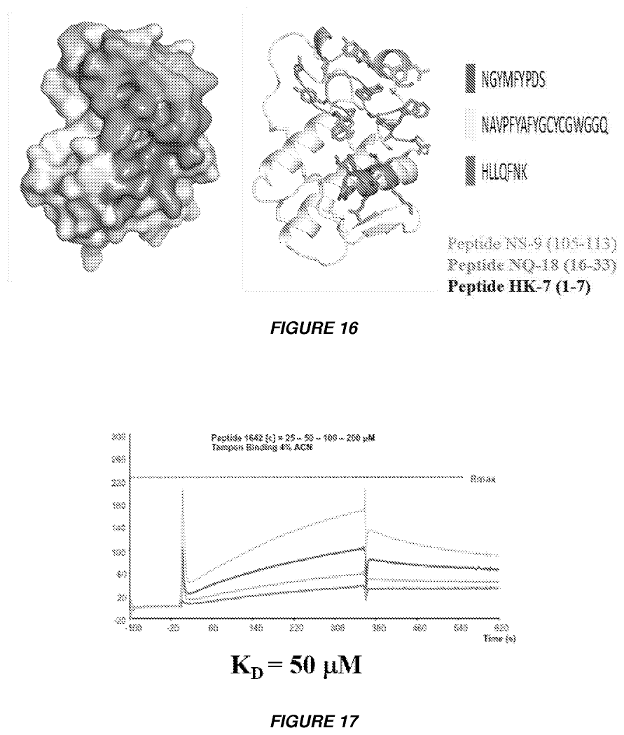

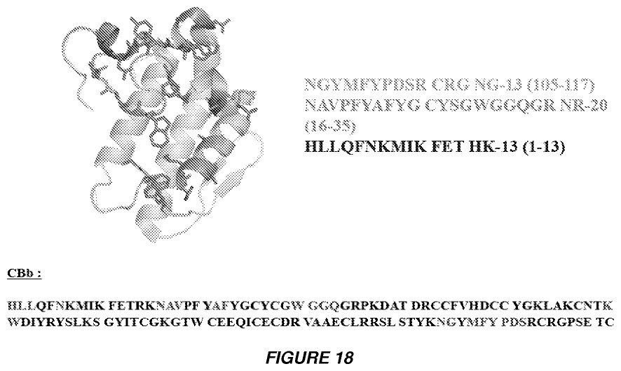

Using surface plasmon resonance (SPR), we investigated the possible interaction between the recombinant, purified hNBD1 of CFTR, covalently attached to a sensor chip, and heterodimeric crotoxin, its nonenzymatic CA subunit and two isoforms (CBa₂ and CBc) of the PLA₂ CB subunit. We demonstrated for the first time that CA-CB complex (FIG. 7), as well as CB alone (isoforms CBc and CBa₂), (FIGS. 1A and B) directly bind to human NBD1. By fitting the 1:1 Langmuir binding model of the experimental association and dissociation curves, the kinetic constants were calculated. The apparent dissociation constant (KDapp) of 118 nM for the CA-CB complex, 4 nM for CBc, and 34 nM for CBa₂ were determined. The two CB isoforms interact with hNBD1 with different kinetics showing the existence of two CB-NBD1 complexes, CBc-NBD1 complex being more stable (FIG. 1B) than that of CBa₂-NBD1 complex (FIG. 1A), the latter dissociating more quickly (Table 1). No binding of the CA subunit to immobilized hNBD1 was observed (FIG. 8A). We also investigated the possible binding of human non-pancreatic hsPLA₂-IIA (FIG. 9A) and pancreatic sPLA₂-IB (FIG. 8C), and observed that only hsPLA₂-IIA, homologous to CB binds to NBD1 (Table 1).

TABLE 1
Kinetic parameters for sPLA₂-NBD1/ΔF508-NBD1 binding
determined by surface plasmone resonance analysis. K_{D}^{app} = k̅_{d}/kₐ.

| PLA₂-NBD1 | kₐ (10⁴ M⁻¹s⁻¹) | k_{d} (10⁻³ s⁻¹) | K_{D}^{app} (nM) |
|---|---|---|---|
| CACB | 4.27 ± 0.5 | 5.1 ± 0.07 | 118 |
| CBc | 5.2 ± 0.8 | 0.22 ± 0.01 | 4 |
| CBa₂ | 91 ± 3 | 31 ± 0.9 | 34 |
| hsPLA₂-IIA | 5.9 ± 0.6 | 2.1 ± 0.03 | 35 |

| PLA₂-ΔF508NBD1 | kₐ (10⁵ M⁻¹s⁻¹) | k_{d} (10⁻³ s⁻¹) | K_{D}^{app} (nM) |
|---|---|---|---|
| CBa₂ | 3.1 ± 0.7 | 8.5 ± 0.04 | 28 |
| hsPLA₂-IIA | 1.8 ± 0.5 | 6.6 ± 0.08 | 37 |

Data are presented as mean ± S.E. of three independent experiments carried out in triplicate

### Example 3: Potentiating Effect of CB on CFTR-Cl— Channel Current

Based on SPR experiments (FIGS. 1A and B, Fig S1A), we hypothesized that CA-CB/NBD1 and CB/NBD1 complexes could modulate CFTR Cl⁻ channel activity. To test this hypothesis, WT-CFTR was expressed in X. laevis oocytes and CFTR-CV current (I_{cAMP}) was activated by superfusing the oocytes with IBMX/Fsk PKA-stimulating cocktail. Subsequently, CA-CB, CA or CB were injected into CFTR-expressing oocytes, and I_{cAMP} were measured after two hours. We observed that I_{cAMP} was significantly increased after injection of CA-CB [50 μg/ml], and inhibited by the specific CFTR-inhibitor Inh₁₇₂, as compared to I_{cAMP} recorded in control oocytes (FIG. 7B, FIG. 7C shows mean results obtained at −100 mV and +40 mV). These electrophysiological data suggest that CA-CB/CFTR interaction is involved in the stimulation of I_{cAMP}.

According to our hypothesis, CA, which does not interact with NBD1 (Fig S2A), should not modulate I_{cAMP}. Our results are consistent with this interpretation, as injection of CA [50 μg/ml] into CFTR-expressing oocytes had no effect on I_{cAMP} (FIG. 8B).

To provide additional evidence that CB, which binds to hNBD1, (FIGS. 1A and B) is involved in modulation of I_{cAMP}, we injected 0.5 ng of CBa₂ into CFTR-expressing oocytes. CBa₂ increased I_{cAMP} (FIGS. 1C and D). Taken together, these results are consistent with an activation of I_{cAMP} due to the interaction of CBa₂ with CFTR.

To investigate if CB increases the expression of CFTR, we performed immunoblotting on microsomal proteins from CFTR-expressing oocytes in the presence or absence of CB (FIG. 1E). Detection of 170 kDa mature CFTR in both conditions (FIG. 1E) shows that coinjection with CBa₂ does not alter CFTR maturation. Altogether, these results suggest that CB may either acutely potentiate I_{cAMP} and/or have a corrector activity.

To test for acute potentiator activity of CB, we performed a series of experiments on HeLa cells expressing WTCFTR. First, we performed current recordings using the inside-out configuration of the patch-clamp technique. A representative experiment is shown in FIG. 2A, and the mean results are shown in FIG. 2B. Addition of 1 nM CB at the intracellular side of the membrane patch resulted in a significant increase in channel activity by a factor of 2.3, after activation of channels of the same single current amplitude by PKA in the presence of 1, 0.5 or 0.05 mM ATP-Mg (n=6) (FIG. 2). These channels were inhibited by glibenclamide and Inh₁₇₂. Addition of CB in the absence of ATP-Mg was without effect (n=3), indicating that CB-induced activation was dependent on the presence of ATP-Mg. These results show a potentiating effect of CB on CFTR.

To verify if CB could be active on living epithelium, we performed short circuit current (Isc) measurements in mouse colon tissue. Addition of 1 μM CB to the basal and apical side of the epithelium after the exposure of tissues increased cAMP-dependent Isc previously activated by Fsk plus IBMx cocktail by 4.9±2.8 (N=6 mice (n=17 colon fragments)). This increase was inhibited by the addition of 100 μM bumetanide, Na—K—Cl inhibitor, to the basolateral side (FIG. 2C). Lower concentrations of CB were without effect. These results are in favor of a potentiating effect of CB in the mouse colon tissue.

### Example 4: Correcting Effect of CB on Mutated ΔF508CFTR

Since the deletion of Phe508 in NBD1 is the most frequent mutation leading to CF, we studied binding of the CB subunit of crotoxin to the ΔF508-NBD1 mutant. FIG. 3A shows the direct binding of CBa₂ isoform to immobilized ΔF508-NBD1 with K_{D}^{app} of 28 nM (Table 1). CBa₂ displays slightly higher affinity for mutated NBD1 (K_{D}^{app} of 27.4±6.5 nM) than for WT (36.7±10.2 nM), but these differences are not statistically significant. The important observation is that kd (the dissociation rate constant) for ΔFNBD1 is 3.6-fold slower than for WTNBD1, suggesting that the binding of CBa₂ to mutated NBD1 stabilizes ΔF508CFTR. We also showed that hGIIA-sPLA₂ binds to immobilized ΔF508-NBD1 (FIG. 10A) with K_{D}^{app} of 37 nM (Table 1).

Because (i) the regulation of CFTR current by CB in X. laevis oocytes was observed two hours after injection of CB, and (ii) CB binds to ΔF508-NBD1, we tested if CB could promote the functional rescue of ΔF508-CFTR in two experimental models (X. laevis oocytes and HeLa cells). In the first series of two electrode voltage clamp experiments, the effect of CBa₂ injection on oocytes expressing ΔF508-CFTR was examined. FIG. 3B shows the I/V ratio for oocytes expressing ΔF508-CFTR alone, and coinjected with ΔF508CFTR-RNA and CBa₂. In both cases, application of IBMX/Fsk stimulating cocktail led to increased I_{cAMP} amplitude. Note that a weak I_{cAMP} value was detected in basal conditions (i.e. in oocytes not co-injected with CBa₂) and a significant increase of channel activity was observed in experimental conditions (i.e. in oocytes co-injected with CBa₂), this increase in I_{cAMP} was smaller than that observed in WT-CFTR-expressing oocytes (compare with FIG. 1C). Inhibition of I_{cAMP} with 10 μM Inh-172 confirmed that the potentiating effect of CBa₂ was related to CFTR function. These data show that CBa₂ increases the Cl⁻ channel activity of mutated CFTR (FIG. 3B). Western blot analysis demonstrated an increase in mature CFTR protein levels in oocytes co-injected with ΔF508-CFTR and CBa₂, suggesting that CB promotes insertion of the mature form of CFTR protein (fully glycosylated band C) and facilitates transport to the plasma membrane (FIG. 3C).

### Example 5: Potentiating Effect of CB on ΔF508CFTR-Cl— Channel Current Previously Rescued by Treatment for 24 h with Other Correctors

In another series of experiments, to test if CB potentiates CFTR currents in ΔF508CFTR, we used ΔF508CFTR expressing HeLa cells pre-treated with correctors and performed nystatin-perforated whole-cell patch-clamp recordings in which ΔF508CFTR was functionally rescued by treatment for 24 h with 1 μM of corr4a or 407 correctors. As shown in FIG. 2D, I_{AMP} recorded after correction was further increased after exposure of cells to InM CB and the current was inhibited by another specific inhibitor of CFTR currents (glyH101), but only slightly by a mixture of two other inhibitors, inh172 and glibenclamide. These results confirmed that CB increases (potentiates) ΔF508CFTR currents previously rescued by treatment for 24 h with other correctors and indicated that binding of CB to CFTR occurs in the cell. CB should mask the inhibitory site of inh172, but not that of glyH101.

### Example 6: Inhibitory Effect of Human hsPLA₂-IIA on ΔF508-CFTR-Cl— Channel Current

Since CB is structurally homologous to human hsPLA₂-IIA and our SPR experiments showed interaction of hsPLA₂-IIA with hNBD1 (WT and mutated) (FIG. 9A, FIG. 10A), we investigated the effect of hsPLA₂-IIA on CFTR function. As summarized in FIG. 9B and S3C, in oocytes co-injected with CFTR-RNA and hsPLA₂, the PKA-activating cocktail increased Inh-172-sensitive I_{cAMP} as compared to oocytes expressing CFTR alone, suggesting that hsPLA₂-IIA regulates CFTR function in a similar manner to CBa₂. However, this effect was not observed after injection of hsPLA₂-IIA into oocytes expressing ΔF508-CFTR. On the contrary, an inhibition of I_{cAMP} was observed (FIG. 10B, 10C). Thus, CBa₂ isoform from snake venom (with potentiating and correcting effects) and hsPLA₂-IIA (with an inhibitory effect) have different effects on ΔF508-CFTR.

### Example 7: Is the Effect of CB on CFTR Independent of the Enzymatic Activity of PLA₂?

Two-electrode voltage-clamp experiments in oocytes and inside-out patches in HeLa cells performed in the absence of calcium suggested that the potentiating effects of CB on CFTR-Cl⁻ channel current were not calcium-dependent. To provide additional evidence that this potentiating effect is independent of the enzymatic activity of PLA₂, we injected into CFTR-expressing oocytes the product of CB enzymatic activity, arachidonic acid. As shown in FIG. 11A, 11B, arachidonic acid (4 μM), had no effect on I_{cAMP} and is not responsible for CFTR functional changes, although it cannot be excluded that a modification of the lipid environment due to PLA₂ activity could play a role in these changes. However, the equimolar substitution of calcium ions by strontium (an inhibitor of PLA₂ activity) does not inhibit the interaction of CB with NBD1 (data not shown), which suggests that the binding effect (and probably potentiating effect) is independent of the enzymatic activity.

### Example 8: Access to the Catalytic Site of CB is Masked by NBD1

To characterize the CB-NBD1 binding interface and to verify if access to the catalytic site of CB is masked by NBD1, we performed two series of experiments, SPR competition experiments and a spectrofluorimetric study using specific PLA₂ inhibitor (PMS 1062)²² to access the inhibitory effect of PMS on PLA₂-NBD1 interaction. As shown in FIG. 12A, when NBD1 was immobilized on the sensor chip, the CB subunit of crotoxin interacted with NBD1, but the CA subunit did not interact with CB, since the CA binding site was occupied by NBD1. In the second series of experiments, the spectrofluorimetric assays showed that PMS 1062 competitively inhibited enzymatic activity of CB alone and CB/NBD1 complex (FIG. 12B). Inhibition constant (Kᵢ) values of 0.025 μM for CB and 0.48 μM for the CB/NBD1 complex were observed. Thus, a twenty-fold higher concentration of the inhibitor is required for inhibition of 50% of PLA₂ activity in the complex, suggesting that access to the catalytic site of PLA₂ is masked by NBD1.

### Example 9: Structural Insight into the Interaction of ΔF508NBD1 with the CB Subunit of Crotoxin

As we showed experimentally, the CB subunit of crotoxin can interact with WT and ΔF508NBD1, forming functional complexes resulting in increased CFTR Cl⁻ channel current (FIG. 1-3). Using SPR experiments, we observed that two isoforms of the CB subunits (CBa₂ and CBc) interact with NBD1 of CFTR with different kinetics (FIGS. 1A and 1B). Two classes of CB/NBD1 complexes were detected (those formed with CBc more stable and dissociating slowly and those formed with CBa₂ less stable and dissociating quickly) (Table 1), similar to the two classes of natural CA-CB complexes described previously²³. We concluded that the mutation His1>Ser, important for stability of crotoxin complexes²⁴, could also be important for stability of CB/NBD1 and CB/ΔF508NBD1 complexes. These data were taken into account in the theoretical calculation of the binding interface between CB and ΔF508NBD1.

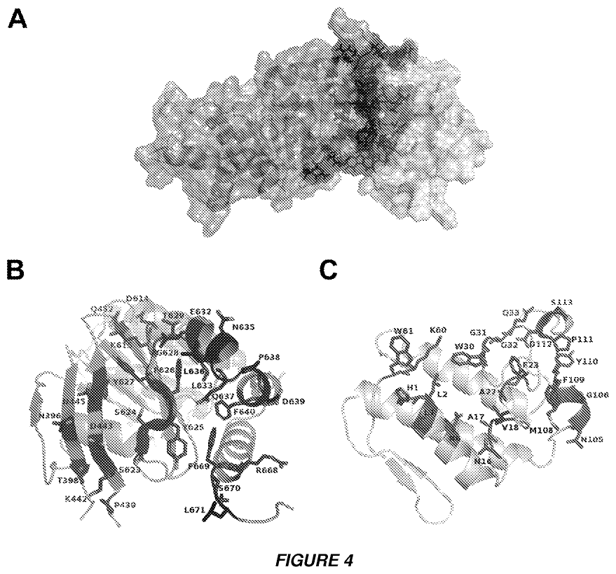

We have applied a molecular docking protocol consisting of the following steps: (a) an initial, rigid body 3D search based on fast a Fourier transform algorithm; (b) primary rescoring with a linear weighted scoring function implemented in ZRANK; (c) structural refinement by Monte Carlo methods; (d) secondary rescoring with ZRANK function optimized for refined complexes. The procedure used here²⁵ has been shown to significantly improve structure prediction of protein-protein complexes. FIG. 4A shows a representation of the highest scored model of the ΔF508NBD1/CB complex structure. The ΔF508NBD1 residues constituting the binding site for the CB subunit mainly derive from three ΔF508-NBD1 subdomains: an anti-parallel 3-sheet subdomain (ABC-β), an F1-type ATP-binding core subdomain and a regulatory extension (RE) fragment (FIG. 4B).

The total solvent accessible surface buried at the ΔF508NBD1/CB interface for the predicted complex is ˜1600 Å²; contribution from the ΔF508NBD1 is ˜786.6 Å² and that from the CB subunit is ˜813.7 Å². The central part of the ΔF508NBD1 interface is characterized by a broad patch of hydrophobic residues including Y625, F626, Y627, L636, F669 and L671 (FIG. 4B), which are in close contact with a complementary patch formed by L2, L3, F24, W31, M118, F119 of the CB subunit (FIG. 4C). Among the more specific interactions are two potential hydrogen bonds between W31-Y625, G33-D443, one ionic interaction between H1 and E632 and two cation-π interactions formed by W70-K615 and K69 and Y627 (Table 2).

TABLE 2
Parameters obtained by molecular docking for ΔF508-NBD1/CBb complex

| Protein-Protein | | | | | | | | | |
|---|---|---|---|---|---|---|---|---|---|
| hydrophobic | | Protein-protein Main chain-Side chain hydrogen bonds | | | | | | | |
| Interaction | | | | | | | | A (d- | A (a- |
| NBD1 | CBb | CBb | | NBD1 | | D d-a | D H-a | H—N) | O═C) |
| Residue | Residue | Residue | Atom | Residue | Atom | [Å] | [Å] | [°] | [°] |
| Tyr 625 | Phe 24 | Trp 31 | NE1 | Tyr 625 | O | 3.49 | 3.34 | 91.65 | 130.65 |
| Phe 626 | Trp31 | Gly 33 | N | Asp 443 | OD2 | 3.21 | 2.25 | 160.64 | 133.24 |

| Tyr 627 | Trp 31 | Protein-protein Side chain-Side chain hydrogen bonds | | | | | | |
|---|---|---|---|---|---|---|---|---|
| Leu 636 | Leu 2 | CBb | Atom | NBD1 | Atom | D d-a | D H-a | A (d- |
| | | Residue | | Residue | | [Å] | [Å] | H—N) |
| | | | | | | | | [°] |
| Leu 636 | Leu 3 | Trp 70 | NE1 | Glu 632 | OE1 | 2.80 | 2.37 | 109.10 |
| Leu 636 | Trp 31 | Trp 70 | NE1 | Glu 632 | OE2 | 3.39 | 2.55 | 154.45 |

| Phe 669 | Phe 119 | Protein-Protein Cation-π Interaction | | | | Protein-Protein Ionic Interaction | |
|---|---|---|---|---|---|---|---|
| Phe 669 | Phe 24 | CBb | NBD1 | DIST | ANGL | CBb | NBD1 |
| | | Residue | Residue | [Å] | [°] | Residue | Residue |
| Leu 671 | Phe 119 | Lys 69 | Tyr 627 | 5.19 | 82.75 | His 1 | Glu 632 |
| Leu 671 | Met 118 | Trp 70 | Lys 615 | 5.51 | 53.67 | | |

| Protein-Protein aromatic-aromatic | | | | Accessible Surface Area |
|---|---|---|---|---|
| Interaction | | | | |
| CBb | NBD1 | D(CC) | DANGL | Buried area upon the complex formation (Å²) 1518.4 |
| Residue | Residue | [Å] | [°] | Buried area upon the complex formation (%) 7.67 |
| Phe24 | Tyr 625 | 4.71 | 60.75 | Interface area (Å²) 759.2 |
| Trp31 | Phe 626 | 5.32 | 98.76 | Interface area NBD1 (%) 5.98 |
| Phe24 | Phe 669 | 5.28 | 113.78 | Interface area CBb (%) 10.69 |
| Phe119 | Phe 669 | 6.65 | 124.41 | POLAR Buried area upon the complex formation ( Å²) 1065.9 |
| | | | | POLAR Buried area upon the complex formation (%) 70.20 |
| | | | | POLAR Interface area (Å²) 532.95 |
| | | | | NO POLAR Buried area upon the complex formation (Å²) 452.4 |
| | | | | NO POLAR Buried area upon the complex formation (%) 29.79 |
| | | | | NO POLAR Interface area (Å²) 226.2 |
| | | | | Residues at the interface - total (n) 44 |
| | | | | Residues at the interface - NBD1 (n) 24 |
| | | | | Residues at the interface - CBb (n) 20 |

D d-a - distance between donor and acceptor,
D H-a - distance between hydrogen and acceptor
A (d-H—N) - angle between donor-H—N
A (a-O═C) - angle between acceptor-O═C
D(CC) - distance (centroid − centroid)
DANGL - dihedral angel
The model of ΔF508-NBD1/CBb complex has been analyzed using Protein Interactions Calculator (http://pic.mbu.iisc.emet.in/) and CoCoMaps (https://www.molnac.unisa.it/BioTools/cocomaps) servers

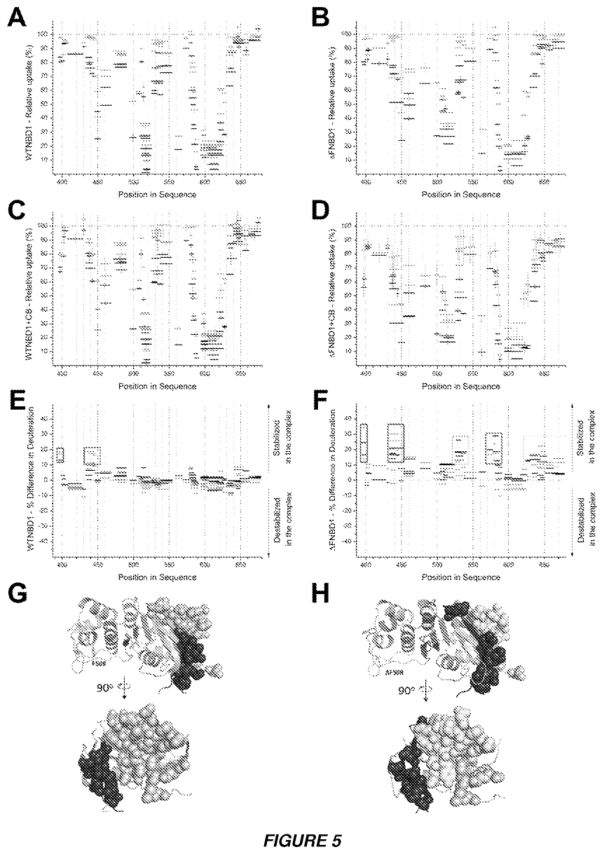

To further characterize the CB-binding sites on WTNBD1/ΔF508NBD1, we performed HDX-MS experiments. The levels of hydrogen-deuterium exchange of amide protons in peptic peptides from WTNBD1 and ΔF508NBD1 were measured in the absence and presence of CB and CA crotoxin subunits. In FIG. 5, the levels of relative deuterium uptake for peptic peptides of WTNBD1 (panels A,C) and ΔF508NBD1 (panels B,D) are shown, both in the absence of CB (panels A,B) and in the presence of CB (panels C,D). Data collected at two incubation times of 10 s (black) and 1 min (orange) are presented. To best visualize the differences we used 1)—subtraction plots, in which the differences in the relative deuterium uptake level (in %) are shown along the protein sequence (FIG. 5 E, F) and 2)—kinetic plots comparing the deuterium uptake at different times of incubation for selected peptides (FIG. 13). While more prominent changes were observed for ΔFNBD1 with

numerous peptides from the same region showing the same direction of change, weaker differences were also observed for WT. This is in agreement with the observations from SPR experiments that WTNBD1-CB complex dissociates much faster as compared to the ΔF508NBD1-CB complex. For WTNBD1, the stabilization is focused in two regions: 395-402, 434-447 (marked by red rectangles in FIG. 5 E and red spheres in panel G). The kinetic plots for two peptides selected from these regions are shown in FIG. 13 (two left panels, marked by the red rectangle) and confirm significant differences in the rates of deuterium uptake. These two regions strictly correspond to the two adjacent strands of the ABC R sheet indicated in molecular docking as a part of the binding site (compare FIG. 4B). This regions also included D443, which according to the model, hydrogen-bonds to G33 of CB. In ΔF508NBD1 (FIG. 5. F,H, FIG. 13), these two regions are also stabilized during complex formation, but the effect is more pronounced and extends to position 452. The binding of CB stabilizes a larger fraction of ABC R in the case of ΔF508NBD1. Also, specifically in ΔFNBD1, the changes observed in ABCβ upon CB-binding are accompanied by the stabilization of other regions, including the Structurally Diverse Region (SDR), spanning positions 526-547 within the ABCa subdomain (marked blue), Walker loop B 572-579 (marked magenta), and C-terminal region (marked yellow) from the F1-like ATP-binding subdomain (620-655) extended into Regulatory Extension (>650) (FIG. 5F, H, FIG. 13). For CA, no such stabilizing effects were observed (FIG. 14). All three segments implicated by molecular docking to constitute the binding surface (395-402, 442-448 of ABCβ, 623-640 of the F1-like ATP-binding subdomain and 668-671 of RE) become stabilized in contact with CB, but not in the presence of CA. Thus, HDX-MS experiments confirm the binding model derived from molecular docking procedures. In the case of ΔF508NBD1, the stabilization is also observed for other regions, namely in ABCa and Walker loop B. These changes most probably indicate ΔF508NBD1-specific allosteric stabilization, caused by the expansion of the binding surface towards the C-terminal part of the NBD1 domain.

### Example 10: CB Interrupts the K8-CFTR Pathogenic Complex

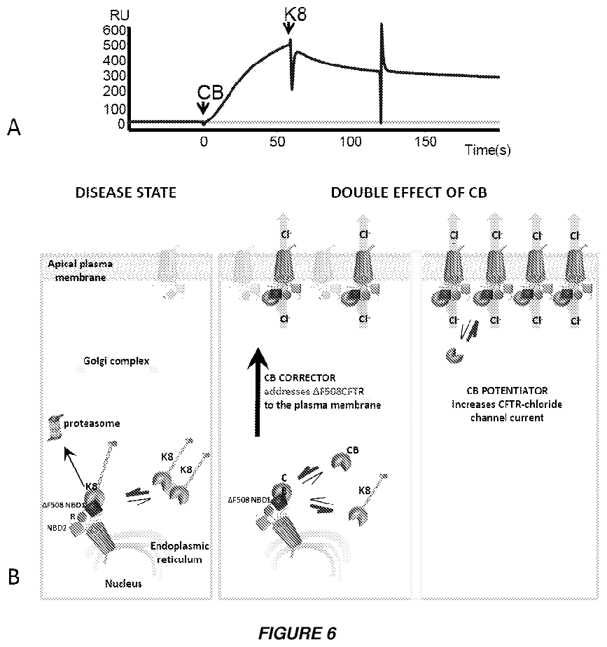

To gain further insight into the mechanisms involved in the interactions between CB and ΔF508CFTR, we performed SPR competition experiments, taking advantage of the known interaction between NBD1/ΔF508NBD1 and keratin 8 (K8)¹⁰. A typical experiment is shown in FIG. 6A. In control experiments, CB and K8 bind to ΔF508NBD1 with nanomolar affinity. We observed that when CB was bound first, the K8 could not interact with NBD1 showing that both proteins interact with NBD1 at similar region(s) (FIG. 6A). We postulate that CB prevents formation of a protein complex between K8 and ΔF508-CFTR (FIG. 6B), and this interruption of the K8-CFTR pathogenic complex triggered by CB allows ΔF508CFTR to escape the degradation pathway and to be delivered to the plasma membrane.

### Example 11: Discussion

This study shows for the first time the direct nanomolar binding of rattlesnake PLA₂ CB to NBD1/ΔF508NBD1 of CFTR and that this interaction has functional consequences. CB behaves as a dual modulator of CFTR activity, it acts as corrector facilitating transport of mutated CFTR to the plasma membrane and as potentiator increasing the Cl⁻ channel current of CFTR. It prompts the question of potential pharmacological applications.

The correcting effect of CB on ΔF508CFTR activity was revealed in two experimental models, X. leavis oocytes and HeLa cells (expressing ΔF508CFTR) using electrophysiological and biochemical assays (FIG. 3). The western blot experiments conducted in HeLa cells showed the maturation of mutated CFTR after treatment with CB. Fully glycosylated band C was detected, (FIG. 3E) reflecting insertion of CFTR into the plasma membrane. Our results show that CB facilitates transport to the membrane of mutated CFTR, but we don't have experimental evidence for insertion of CB-ΔF508CFTR complex into the membrane. The mechanism at cellular level needs more investigation and the hypothesis that the bound CB moiety has to cross the membrane will need to be examined in the future. The amplitude of CFTR current induced by CB correction was even larger than that induced by Corr4A⁹ showing efficient benefit of the CB-ΔF508CFTR interaction. Human hsPLA₂-IIA, homologous to CB, which did not increase DF508CFTR Cl⁻ current but even reduced its activity showed that binding of hsPLA₂-IIA to ΔF508NBD1 has negative functional consequences on CFTR current. It further underlines the choice of snake venom PLA₂ as an adequate template for the future structure-based design of new CFTR modulators.

Here, we present the first demonstration for a beneficial protein-protein interaction, CB-ΔF508CFTR, which leads to correction of the functional defect of ΔF508-CFTR (FIG. 6). CB behaves as a competitor of K8 for binding to ΔF508NBD1 known to form a pathogenic complex preventing the delivery of ΔF508CFTR to the plasma membrane^{9; 10}. It further supports the hypothesis that PLA₂-ΔF508CFTR interaction is a therapeutic target for ΔF508 patients.

CB is not only a corrector, but also acts as a potentiator of CFTR Cl⁻ currents, fitting into a new class of modulators encompassing both correcting and potentiating activities²⁶. The potentiating activity of CB was demonstrated in different cell models and in mouse colon tissue by electrophysiological assays (FIG. 1, 2). In all assays, significant increases of CFTR currents were recorded. The experiments using inside-out configuration of the patch-clamp technique confirmed that exposure of the intracellular side of the membrane patch to CB resulted in a significant increase (by a factor of 2.3) of channel activity. This effect is dependent on ATP. HDXMS confirmed that Walker loop B and F1-like ATP-binding subdomain are involved in the CB-ΔF508NBD1 interaction. The physiological relevance of the potentiating effect is strengthened by the observation ex-vivo in mouse colon tissue (FIG. 2C).

Determination of the regions in CB responsible for functional effects may represent a fundamental step in the development of a new dual potentiator and corrector for DF508CFTR. To identify the binding interface between CB and NBD1, we studied the interaction of heterodimeric crotoxin complex with DF508NBD1 and observed that the affinity of the PLA₂ subunit alone for hNBD1 is 30 times higher than that of the CA-CB complex. Even if the CA subunit does not directly interact with NBD1, it partially inhibits interaction between CB and NBD1 suggesting similar binding interfaces between CA-CB and CB/NBD1. Kinetic analyses revealed a critical mutation H1/S1CB²⁴ located in the CB-NBD1 binding interface supporting our hypothesis. The fluorometric analysis showed that the access to the catalytic site of PLA₂ is masked by NBD1.

Using molecular docking simulation and biophysical experimental data, we proposed a structural 3D model of the DF508NBD1/CBb complex (FIG. 4). This model shows that the binding interfaces between CA-CB and those of CB/DF508NBD1 significantly overlap (FIG. 12C). Similar to the crystal structure of CA-CB complex, the access to the crucial PLA₂ catalytic residues H48 and D99 is restricted by aromatic residues, which may participate in binding to DF508NBD1. We propose that the CB/DF508NBD1 interface is composed of several interacting hydrophobic residues and a few polar contacts (among them, the salt bridge between E632 of DF508NBD1 and H1 of the CBb subunit). In isoform CBa₂, H1 is replaced by Si, which deprives the interface of its only ionic interaction, potentially leading to differences in binding affinity between CBa₂ and CBc with NBD1 (FIGS. 1A and B).

The binding regions at ΔF508NBD1 proposed by molecular docking of ΔF508NBD1 and CB were confirmed in the HDX-MS experiments. For ΔF508NBD1, these experiments showed retarded exchange of amide protons in the peptides belonging to the F1-like ATP binding core sub-domain (boxed in yellow in FIG. 5F and FIG. 13) containing hydrophobic residues (Y625, F626, Y627, L636) and the ABCB subdomain (boxed in red in FIG. 5F and Supplementary FIG. 13) containing D443 and identified by molecular docking calculations as contact residues (see Table 2). The latter region was also found to be more protected in the complex with WTNBD1 (FIG. 5E,G and Supplementary FIG. 13). In ΔF508-NBD1 in general a more widespread stabilization was observed with two more regions undergoing stabilization, most probably representing allosteric changes caused by CB binding to the F1-like ATP binding core sub-domain. The first corresponds to a helix at positions 526-547 within the ABCa subdomain and the second in Walker loop B. FIG. 5 shows the superposition of the ΔF508NBD1 binding interface: residues identified by the theoretical calculation vs. HDX-MS experiment, showing overlapping and good experimental confirmation of our 3D molecular model.

Our results open an exciting field of investigation as they evoke new and unexpected functions of secreted sPLA2 CB from rattlesnake venom. Since it has been proposed that effective pharmacotherapy could be based on a combination of two different types of drugs, correctors and potentiators in order to address trafficking and gating defects, respectively 27, the double effect of CB as corrector and potentiator described here provides an original perspective to develop new therapeutic molecules. The interface identified between rattlesnake sPLA₂ and ΔF508NBD1 constitutes a promising target in the development of new anti-CF agents.

### Example 12

Peptides of SEQ ID NOS: 1-3 derived from CB and designed in accordance with the CB-DF508NBD1 model have been assessed for their interaction with the NBD1 domain of DF508CFTR. These peptides were shown to bind with mutated NBD1. The SPR profile has shown interaction of peptide having SEQ ID NO: 2 (NQ-18(16-33)) with DF508-NBD1 and has shown that CL-channel current is increased (FIG. 17). Electrophysiological tests are currently ongoing.

Three alternative peptides with slightly different sequences (having the sequences of SEQ ID NOS: 4, 5 and 6) have been designed and synthesized; they are shown on FIG. 18 which provides their location in the CBb unit (SEQ ID NO: 7). Their interaction is assessed by SPR. One of these peptides i.e., NG-13(105-117) having the sequence of SEQ ID NO: 4 has proved to be soluble and able to bind mutated NBD1.

### REFERENCES

1. Lewis, H. A., Wang, C., Zhao, X., Hamuro, Y., Conners, K., Kearins, M. C., Lu, F., Sauder, J. M., Molnar, K. S., Coales, S. J., Maloney, P. C., Guggino, W. B., Wetmore, D. R., Weber, P. C. & Hunt, J. F. (2010). Structure and dynamics ofNBD1 from CFTR characterized using crystallography and hydrogen/deuterium exchange mass spectrometry. J Mol Biol 396, 406-30.
2. Kirk, K. L. & Wang, W. (2011). A unified view of cystic fibrosis transmembrane conductance regulator (CFTR) gating: combining the allosterism of a ligand-gated channel with the enzymatic activity of an ATP-binding cassette (ABC) transporter. J Biol Chem 286, 12813-9.
3. Ollero, M., Brouillard, F. & Edelman, A. (2006). Cystic fibrosis enters the proteomics scene: new answers to old questions. Proteomics 6, 4084-99.
4. Pranke, I. M. & Sermet-Gaudelus, I. (2014). Biosynthesis of cystic fibrosis transmembrane conductance regulator. Int J Biochem Cell Biol 52, 26-38.
5. Dalemans, W., Barbry, P., Champigny, G., Jallat, S., Dott, K., Dreyer, D., Crystal, R. G., Pavirani, A., Lecocq, J. P. & Lazdunski, M. (1991). Altered chloride ion channel kinetics associated with the delta F508 cystic fibrosis mutation. Nature 354, 526-8.
6. Becq, F., Mall, M. A., Sheppard, D. N., Conese, M. & Zegarra-Moran, O. (2011). Pharmacological therapy for cystic fibrosis: from bench to bedside. J Cyst Fibros 10 Suppl 2, S129-45.
7. Lipecka, J., Norez, C., Bensalem, N., Baudouin-Legros, M., Planelles, G., Becq, F., Edelman, A. & Davezac, N. (2006). Rescue of DeltaF508-CFTR (cystic fibrosis transmembrane conductance regulator) by curcumin: involvement of the keratin 18 network. J Pharmacol Exp Ther 317, 500-5.
8. Hamdaoui, N., Baudoin-Legros, M., Kelly, M., Aissat, A., Moriceau, S., Vieu, D. L., Colas, J., Fritsch, J., Edelman, A. & Planelles, G. (2011). Resveratrol rescues cAMP-dependent anionic transport in the cystic fibrosis pancreatic cell line CFPAC 1. Br J Pharmacol 163, 876-86.
9. Odolczyk, N., Fritsch, J., Norez, C., Servel, N., da Cunha, M. F., Bitam, S., Kupniewska, A., Wiszniewski, L., Colas, J., Tarnowski, K., Tondelier, D., Roldan, A., Saussereau, E. L., Melin-Heschel, P., Wieczorek, G., Lukacs, G. L., Dadlez, M., Faure, G., Herrmann, H., Ollero, M., Becq, F., Zielenkiewicz, P. & Edelman, A. (2013). Discovery of novel potent DeltaF508-CFTR correctors that target the nucleotide binding domain. EMBO Mol Med 5, 1484-501.
10. Colas, J., Faure, G., Saussereau, E., Trudel, S., Rabeh, W. M., Bitam, S., Guerrera, I. C., Fritsch, J., Sermet-Gaudelus, I., Davezac, N., Brouillard, F., Lukacs, G. L., Herrmann, H., Ollero, M. & Edelman, A. (2012). Disruption of cytokeratin-8 interaction with F508del-CFTR corrects its functional defect. Hum Mol Genet 21, 623-34.
11. Rigoni, M., Paoli, M., Milanesi, E., Caccin, P., Rasola, A., Bernardi, P. & Montecucco, C. (2008). Snake phospholipase A2 neurotoxins enter neurons, bind specifically to mitochondria, and open their transition pores. J Biol Chem 283, 34013-20.
12. Sribar, J. & Krizaj, I. (2011). Secreted Phospholipases A2—not just Enzymes. Acta Chim Slov 58, 678-688.
13. Faure, G., Gowda, V. T. & Maroun, R. C. (2007). Characterization of a human coagulation factor Xa-binding site on Viperidae snake venom phospholipases A2 by affinity binding studies and molecular bioinformatics. BMC Struct Biol 7, 82.
14. Faure, G., Xu, H. & Saul, F. A. (2010). Anticoagulant phospholipases A2 which bind to the specific soluble receptor Coagulation Factor Xa. In Toxins and Hemostasis: From Bench to Bedside (Kini, R. M., Clemetson, K., Markland, M. A. & Morita, T., eds.). Springer Science+Business Media B. V.
15. Petrovic, U., Sribar, J., Paris, A., Rupnik, M., Krzan, M., Vardjan, N., Gubensek, F., Zorec, R. & Krizaj, I. (2004). Ammodytoxin, a neurotoxic secreted phospholipase A(2), can act in the cytosol of the nerve cell. Biochem Biophys Res Commun 324, 981-5.
16. Faure, G. & Bon, C. (1988). Crotoxin, a phospholipase A2 neurotoxin from the South American rattlesnake Crotalus durissus terrificus: purification of several isoforms and comparison of their molecular structure and of their biological activities. Biochemistry 27, 730-8.
17. Cura, J. E., Blanzaco, D. P., Brisson, C., Cura, M. A., Cabrol, R., Larrateguy, L., Mendez, C., Sechi, J. C., Silveira, J. S., Theiller, E., de Roodt, A. R. & Vidal, J. C. (2002). Phase I and pharmacokinetics study of crotoxin (cytotoxic PLA(2), NSC-624244) in patients with advanced cancer. Clin Cancer Res 8, 1033-41.
18. Wang, J., Qin, X., Zhang, Z., Chen, M., Wang, Y. & Gao, B. (2014). Crotoxin suppresses the tumorigenic properties and enhances the antitumor activity of Iressa® (gefinitib) in human lung adenocarcinoma SPCA1 cells. Mol Med Rep 10, 3009-14.
19. Zhang, P., Lader, A. S., Etcheverry, M. A. & Cantiello, H. F. (2010). Crotoxin potentiates L-type calcium currents and modulates the action potential of neonatal rat cardiomyocytes. Toxicon 55, 1236-43.
20. Lomeo Rda, S., Goncalves, A. P., da Silva, C. N., de Paula, A. T., Costa Santos, D. O., Fortes-Dias, C. L., Gomes, D. A. & de Lima, M. E. (2014). Crotoxin from Crotalus durissus terrificus snake venom induces the release of glutamate from cerebrocortical synaptosomes via N and P/Q calcium channels. Toxicon 85, 5-16.
21. Ollivier-Bousquet, M., Radvanyi, F. & Bon, C. (1991). Crotoxin, a phospholipase A2 neurotoxin from snake venom, interacts with epithelial mammary cells, is internalized and induces secretion. Mol Cell Endocrinol 82, 41-50.
22. Assogba, L., Ahamada-Himidi, A., Habich, N. M., Aoun, D., Boukli, L., Massicot, F., Mounier, C. M., Huet, J., Lamouri, A., Ombetta, J. E., Godfroid, J. J., Dong, C. Z. & Heymans, F. (2005). Inhibition of secretory phospholipase A2. 1-design, synthesis and structure-activity relationship studies starting from 4-tetradecyloxybenzamidine to obtain specific inhibitors of group II sPLA2s. Eur J Med Chem 40, 850-61.
23. Faure, G., Harvey, A. L., Thomson, E., Saliou, B., Radvanyi, F. & Bon, C. (1993). Comparison of crotoxin isoforms reveals that stability of the complex plays a major role in its pharmacological action. Eur J Biochem 214, 491-6.
24. Faure, G., Xu, H. & Saul, F. A. (2011). Crystal structure of crotoxin reveals key residues involved in the stability and toxicity of this potent heterodimeric beta-neurotoxin. J Mol Biol412, 176-91.
25. Pierce, B. & Weng, Z. (2008). A combination of rescoring and refinement significantly improves protein docking performance. Proteins 72, 270-9.
26. Favia, M., Mancini, M. T., Bezzerri, V., Guerra, L., Laselva, O., Abbattiscianni, A. C., Debellis, L., Reshkin, S. J., Gambari, R., Cabrini, G. & Casavola, V. (2014). Trimethylangelicin promotes the functional rescue of mutant F508del CFTR protein in cystic fibrosis airway cells. Am J Physiol Lung Cell Mol Physiol 307, L48-61.
27. Pedemonte, N. & Galietta, L. J. (2012). Pharmacological Correctors of Mutant CFTR Mistrafficking. Front Pharmacol 3, 175.
28. Mounier, C. M., Hackeng, T. M., Schaeffer, F., Faure, G., Bon, C. & Griffin, J. H. (1998). Inhibition of prothrombinase by human secretory phospholipase A2 involves binding to factor Xa. J Biol Chem 273, 23764-72.
29. Cougnon, M., Benammou, S., Brouillard, F., Hulin, P. & Planelles, G. (2002). Effect of reactive oxygen species on NH4+ permeation in Xenopus laevis oocytes. Am J Physiol Cell Physiol 282, C1445-53.
30. Lewarchik, C. M., Peters, K. W., Qi, J. & Frizzell, R. A. (2008). Regulation of CFTR trafficking by its R domain. J Biol Chem 283, 28401-12.
31. Nagel, G., Barbry, P., Chabot, H., Brochiero, E., Hartung, K. & Grygorczyk, R. (2005). CFTR fails to inhibit the epithelial sodium channel ENaC expressed in Xenopus laevis oocytes. J Physiol 564, 671-82.
32. Taddei, A., Folli, C., Zegarra-Moran, O., Fanen, P., Verkman, A. S. & Galietta, L. J. (2004). Altered channel gating mechanism for CFTR inhibition by a high-affinity thiazolidinone blocker. FEBS Lett 558, 52-6.
33. Clarke, L. L. (2009). A guide to Ussing chamber studies of mouse intestine. Am J Physiol Gastrointest Liver Physiol 296, G1151-66.
34. Lourdel, S., Paulais, M., Cluzeaud, F., Bens, M., Tanemoto, M., Kurachi, Y., Vandewalle, A. & Teulon, J. (2002). An inward rectifier K(+) channel at the basolateral membrane of the mouse distal convoluted tubule: similarities with Kir4-Kir5.1 heteromeric channels. J Physiol 538, 391-404.
35. Rabeh, W. M., Bossard, F., Xu, H., Okiyoneda, T., Bagdany, M., Mulvihill, C. M., Du, K., di Bernardo, S., Liu, Y., Konermann, L., Roldan, A. & Lukacs, G. L. (2012). Correction of both NBD1 energetics and domain interface is required to restore DeltaF508 CFTR folding and function. Cell 148, 150-63.
36. Radvanyi, F., Jordan, L., Russo-Marie, F. & Bon, C. (1989). A sensitive and continuous fluorometric assay for phospholipase A2 using pyrene-labeled phospholipids in the presence of serum albumin. Anal Biochem 177, 103-9.
37. Dixon, M. (1953). The determination of enzyme inhibitor constants. Biochem J 55, 170-1.
38. Lewis, H. A., Zhao, X., Wang, C., Sauder, J. M., Rooney, I., Noland, B. W., Lorimer, D., Kearins, M. C., Conners, K., Condon, B., Maloney, P. C., Guggino, W. B., Hunt, J. F. & Emtage, S. (2005). Impact of the deltaF508 mutation in first nucleotide-binding domain of human cystic fibrosis transmembrane conductance regulator on domain folding and structure. J Biol Chem 280, 1346-53.
39. Bernstein, F. C., Koetzle, T. F., Williams, G. J., Meyer, E. F., Jr., Brice, M. D., Rodgers, J. R., Kennard, O., Shimanouchi, T. & Tasumi, M. (1977). The Protein Data Bank. A computer-based archival file for macromolecular structures. Eur J Biochem 80, 319-24.
40. Pierce, B. G., Hourai, Y. & Weng, Z. (2011). Accelerating protein docking in ZDOCK using an advanced 3D convolution library. PLoS One 6, e24657.
41. Pierce, B. & Weng, Z. (2007). ZRANK: reranking protein docking predictions with an optimized energy function. Proteins 67, 1078-86.
42. Leaver-Fay, A., Tyka, M., Lewis, S. M., Lange, O. F., Thompson, J., Jacak, R., Kaufman, K., Renfrew, P. D., Smith, C. A., Sheffler, W., Davis, I. W., Cooper, S., Treuille, A., Mandell, D. J., Richter, F., Ban, Y. E., Fleishman, S. J., Corn, J. E., Kim, D. E., Lyskov, S., Berrondo, M., Mentzer, S., Popovic, Z., Havranek, J. J., Karanicolas, J., Das, R., Meiler, J., Kortemme, T., Gray, J. J., Kuhlman, B., Baker, D. & Bradley, P. (2011). ROSETTA3: an object-oriented software suite for the simulation and design of macromolecules. Methods Enzymol 487, 545-74.
43. Serohijos, A. W., Hegedus, T., Aleksandrov, A. A., He, L., Cui, L., Dokholyan, N. V. & Riordan, J. R. (2008). Phenylalanine-508 mediates a cytoplasmic-membrane domain contact in the CFTR 3D structure crucial to assembly and channel function. Proc Natl Acad Sci USA 105, 3256-61.
44. Tina, K. G., Bhadra, R. & Srinivasan, N. (2007). PIC: Protein Interactions Calculator. Nucleic Acids Res 35, W473-6.
45. DeLano, W. L. (2002). The Pymol Molecular Graphic System, pp. http://www.pymol.org.
46. Bakouh, N., Cherif-Zahar, B., Hulin, P., Prie, D., Friedlander, G., Edelman, A. & Planelles, G. (2012). Functional interaction between CFTR and the sodium-phosphate co-transport type 2a in Xenopus laevis oocytes. PLoS One 7, e34879.

1. A method of increasing CFTR activity in a cell, comprising contacting the cell with a peptide modulator of CFTR to thereby increase CFTR activity in the cell; wherein the peptide modulator comprises or consists of an amino acid fragment of the CB subunit of crotoxin from Crotalus durrissus terrificus venom.

2. The method of claim 1, wherein the peptide modulator binds to the nucleotide binding domain 1 (NBD1) of CFTR.

3. The method of claim 1, wherein binding of the peptide modulator to CFTR increases CFTR activity by increasing Cl⁻ channel current in a cell comprising the CFTR.

4. The method of claim 1, wherein binding of the peptide modulator to CFTR increases CFTR activity by increasing the plasma membrane fraction of CFTR in a cell comprising the CFTR.

5. The method of claim 1, wherein binding of the peptide modulator to CFTR increases CFTR activity by increasing Cl⁻ channel current in a cell comprising the CFTR and increasing the plasma membrane fraction of CFTR in a cell comprising the CFTR.

6. The method of claim 1, wherein the CFTR is ΔF508CFTR.

7. The method of claim 1, wherein the peptide modulator is selected from:
a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1;
a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and
a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3.

8. The method of claim 1, wherein the peptide modulator is selected from:
a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4;
a polypeptide comprising the amino acid sequence NAVPFYAFYGCYSGWGGQGR (SEQ ID NO: 5), a polypeptide consisting of the amino acid sequence SEQ ID NO: 5; and a polypeptide comprising a functional variant of SEQ ID NO: 5; and
a polypeptide comprising the amino acid sequence HLLQFNKMIKFET (SEQ ID NO: 6), a polypeptide consisting of the amino acid sequence SEQ ID NO: 6, and a polypeptide comprising a functional variant of SEQ ID NO: 6.

9. The method of claim 1, wherein the peptide modulator comprises a chemical modification.

10. A method of treating cystic fibrosis in a subject in need thereof, comprising administering an effective amount of a peptide modulator of CFTR to the subject to thereby increase CFTR activity in the subject; wherein the peptide modulator comprises or consists of an amino acid fragment of the CB subunit of crotoxin from Crotalus durrissus terrificus venom.

11. The method of claim 10, wherein the peptide modulator binds to the nucleotide binding domain 1 (NBD1) of CFTR.

12. The method of claim 10, wherein binding of the peptide modulator to CFTR increases CFTR activity by increasing Cl⁻ channel current in a cell comprising the CFTR.

13. The method of claim 10, wherein binding of the peptide modulator to CFTR increases CFTR activity by increasing the plasma membrane fraction of CFTR in a cell comprising the CFTR.

14. The method of claim 10, wherein binding of the peptide modulator to CFTR increases CFTR activity by increasing Cl⁻ channel current in a cell comprising the CFTR and increasing the plasma membrane fraction of CFTR in a cell comprising the CFTR.

15. The method of claim 10, wherein the CFTR is ΔF508CFTR.

16. The method of claim 10, wherein the peptide modulator is selected from:
a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1;
a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and
a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3.

17. The method of claim 10, wherein the peptide modulator is selected from:
a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4;
a polypeptide comprising the amino acid sequence NAVPFYAFYGCYSGWGGQGR (SEQ ID NO: 5), a polypeptide consisting of the amino acid sequence SEQ ID NO: 5; and
a polypeptide comprising a functional variant of SEQ ID NO: 5; and
a polypeptide comprising the amino acid sequence HLLQFNKMIKFET (SEQ ID NO: 6), a polypeptide consisting of the amino acid sequence SEQ ID NO: 6, and
a polypeptide comprising a functional variant of SEQ ID NO: 6.

18. The method of claim 10, wherein the peptide modulator comprises a chemical modification.

19. A pharmaceutical composition comprising a peptide modulator of CFTR; wherein the peptide modulator comprises or consists of an amino acid fragment of the CB subunit of crotoxin from Crotalus durrissus terrificus venom.

20. The pharmaceutical composition of claim 19, wherein the peptide modulator binds to the nucleotide binding domain 1 (NBD1) of CFTR.

21. The pharmaceutical composition of claim 19, wherein binding of the peptide modulator to CFTR increases CFTR activity by increasing Cl⁻ channel current in a cell comprising the CFTR.

22. The pharmaceutical composition of any one of claim 19, wherein binding of the peptide modulator to CFTR increases CFTR activity by increasing the plasma membrane fraction of CFTR in a cell comprising the CFTR.

23. The pharmaceutical composition of claim 19, wherein binding of the peptide modulator to CFTR increases CFTR activity by increasing Cl⁻ channel current in a cell comprising the CFTR and increasing the plasma membrane fraction of CFTR in a cell comprising the CFTR.

24. The pharmaceutical composition of claim 19, wherein the CFTR is ΔF508CFTR.

25. The pharmaceutical composition of claim 19, wherein the peptide modulator is selected from:
a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1;
a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and
a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3.

26. The pharmaceutical composition of claim 19, wherein the peptide modulator is selected from:
a polypeptide comprising the amino acid sequence NGYMFYPDSRCRG (SEQ ID NO: 4); a polypeptide consisting of the amino acid sequence SEQ ID NO: 4, and a polypeptide comprising a functional variant of SEQ ID NO: 4;
a polypeptide comprising the amino acid sequence NAVPFYAFYGCYSGWGGQGR (SEQ ID NO: 5), a polypeptide consisting of the amino acid sequence SEQ ID NO: 5; and
a polypeptide comprising a functional variant of SEQ ID NO: 5; and
a polypeptide comprising the amino acid sequence HLLQFNKMIKFET (SEQ ID NO: 6), a polypeptide consisting of the amino acid sequence SEQ ID NO: 6, and
a polypeptide comprising a functional variant of SEQ ID NO: 6.

27. The pharmaceutical composition of claim 19, wherein the peptide modulator comprises a chemical modification.

28. (canceled)

29. (canceled)

30. A method of in vitro characterizing a CFTR modulator, comprising:
contacting a cell that expresses CFTR with a peptide modulator of CFTR that increases CFTR activity in the cell;
contacting the cell with a candidate agent; and
determining whether the candidate agent modulates the effect of the peptide modulator of CFTR on CFTR activity;
wherein the peptide modulator comprises or consists of an amino acid fragment of the CB subunit of crotoxin from Crotalus durrissus terrificus venom.

31. The method of claim 30, wherein the peptide modulator is selected from:
a polypeptide comprising the amino acid sequence HLLQFNK (SEQ ID NO: 1), a polypeptide consisting of the amino acid sequence SEQ ID NO: 1, and a polypeptide comprising a functional variant of SEQ ID NO: 1;
a polypeptide comprising the amino acid sequence NAVPFYAFYGCYCGWGGQ (SEQ ID NO: 2), a polypeptide consisting of the amino acid sequence SEQ ID NO: 2, and a polypeptide comprising a functional variant of SEQ ID NO: 2; and
a polypeptide comprising the amino acid sequence NGYMFYPDS (SEQ ID NO: 3), a polypeptide consisting of the amino acid sequence SEQ ID NO: 3, and a polypeptide comprising a functional variant of SEQ ID NO: 3.

32. The method of claim 30, wherein the peptide modulator is selected from:
a polypeptide comprising the amino acid sequence NGYMFYPDSRCRG (SEQ ID NO: 4); a polypeptide consisting of the amino acid sequence SEQ ID NO: 4, and
a polypeptide comprising a functional variant of SEQ ID NO: 4;
a polypeptide comprising the amino acid sequence NAVPFYAFYGCYSGWGGQGR (SEQ ID NO: 5), a polypeptide consisting of the amino acid sequence SEQ ID NO: 5; and
a polypeptide comprising a functional variant of SEQ ID NO: 5; and
a polypeptide comprising the amino acid sequence HLLQFNKMIKFET (SEQ ID NO: 6), a polypeptide consisting of the amino acid sequence SEQ ID NO: 6, and
a polypeptide comprising a functional variant of SEQ ID NO: 6.

33. The method of claim 30, wherein the candidate agent modulates the effect of the peptide modulator of CFTR on CFTR activity and the candidate agent is identified as a CFTR modulator.
